(19) United States
(12) Patent Application Publication
(10) Pub. No.: US 2026/0217772 A1
(43) Pub. Date: Jul. 30, 2026

(54) ENGINEERED PARAMYXOVIRUS SOLUBLE FUSION (F) PROTEINS AND RELATED VACCINES

(71) Applicant: THE SCRIPPS RESEARCH INSTITUTE, La Jolla, CA (US)

(72) Inventors: Linling HE, San Diego, CA (US); Jiang ZHU, San Diego, CA (US); Ian WILSON, La Jolla, CA (US); Yi-Zong LEE, San Diego, CA (US)

(21) Appl. No.: 19/146,567

(22) PCT Filed: Jan. 16, 2024
(86) PCT No.: PCT/US2024/011566

§ 371 Date: Jul. 9, 2025

Related U.S. Application Data
(60) Provisional application No. 63/480,261, filed on Jan. 17, 2023.
(60) Provisional application No. 63/488,985, filed on Mar. 8, 2023.

(51) Int. Cl. C07K 14/135; A61K 31/7088; A61K 38/00; A61K 39/00; A61K 39/155; C12N 15/62

(52) U.S. Cl. CPC C07K 14/135; A61K 31/7088; A61K 38/00; A61K 39/155; C12N 15/62; A61K 2039/55555; C07K 2319/00; C12N 2760/18522; C12N 2760/18534

(57) ABSTRACT

The present invention provides engineered soluble F proteins of paramyxoviruses such as respiratory syncytial viruses (RSVs), human metapneumoviruses (hMPVs), and human parainfluenza viruses (hPIVs). These engineered proteins are stabilized via specific modifications in the wildtype soluble F sequences, e.g., substitutions in the 023 strand and/or introducing an engineered disulfide bond in a 0 hairpin in the F1 subunit. Also provided in the invention are nanoparticle vaccines that contain the engineered soluble F immunogens displayed on self-assembling nanoparticles. The invention also provides methods of using such vaccine compositions in various therapeutic applications, e.g., for preventing or treating viral infections such as RSV, MPV and PIV infections.

### CROSS-REFERENCE TO RELATED APPLICATIONS

The subject patent application claims the benefit of priority to U.S. Provisional Patent Application Nos. 63/480,261 (filed Jan. 17, 2023; now pending) and 63/488,985 (filed Mar. 8, 2023; now pending). The full disclosures of the priority applications are incorporated herein by reference in their entirety and for all purposes.

### BACKGROUND OF THE INVENTION

Respiratory syncytial virus (RSV), human metapneumovirus (hMPV), and parainfluenza viruses (PIVs) are enveloped, non-segmented, negative-sense, single-stranded RNA viruses belonging to the Paramyxoviridae family. Among these, RSV has been studied extensively. The RSV genome encodes three envelope glycoproteins and eight non-structural proteins (NS1, NS2, N, P, M, M2-1, M2-2 and L). The three envelope glycoproteins are the attachment (G) protein, the fusion (F) protein, and the small hydrophobic (SH) protein. F and G are crucial for RSV infectivity and pathogenesis and carry various antigenic determinants that can be recognized by the host neutralizing antibodies (NAbs). As highlighted in the 2015-2016 reports by WHO and Gates Foundation and in a global survey, RSV is a prevalent cause of acute lower respiratory infection (ALRI) in neonates and infants and constitutes a major health burden to developing countries. RSV causes acute respiratory infection, accounting for ~66,000-200,000 deaths and 3.5 million hospitalizations worldwide in children under 5 years of age.

A series of advancements has been made in RSV vaccine development over the last decade. First, a structural understanding has been achieved for the F protein at both prefusion and postfusion states and for RSV neutralization by F-specific NAbs. The F protein mediates viral entry and is a primary target for vaccine development. Multiple antigenic sites (AS) on the F protein can be recognized by NAbs. Co-crystallization of NAb D25 with F resulted in the first atomic structure of prefusion F and revealed a novel antigenic site (AS-Ø) near the trimeric apex, which is composed of residues 62-69 of F2 and the solvent-exposed portion (α4-helix) of F. This structure has enabled the design of prefusion-stabilizing mutations and the structural analysis of other NAbs such as AM1429 and Ø-specific 5C4. Second, both epitope- and F protein-based strategies have been explored in RSV vaccine development. In an early study, immunogen design by ‘epitope grafting’ was demonstrated for the RSV Motavizumab epitope, which led to a proof-of-concept study of an RSV epitope vaccine. Various empirical designs with different sets of mutations have been proposed to stabilize the prefusion F structure. Third, recent human vaccine trials revealed the importance of the prefusion F in NAb elicitation. Compared to the failed postfusion F vaccine, the rationally designed prefusion F trimer (DS-Cav1) showed a 10-fold higher serum NAb response. However, despite these advancements, current RSV prefusion F designs have various problems. For example, DS-Cav1 showed a poor expression profile with a lot of aggregates and other F species after D25 purification. Similarly, another major vaccine candidate, SC-TM, barely had any trimer yield. In the negative-stain EM analysis, while DS-Cav1 appeared to be entirely monomeric, SC-TM showed closed prefusion trimers mixed with postfusion trimers. A further optimized DS-Cav1 design, termed sc9-10 DS-Cav1, included an inter-protomer disulfide bond to lock F in a completely closed trimer conformation. However, such inter-protomer disulfide bonds could disrupt the folding of F-nanoparticle (NP) protein and assembly of F-presenting NPs, resulting in low yield and protein aggregation. In addition, sc9-10 DS-Cav1 contains many mutations generated by random mutagenesis that may or may not be essential to the structure and function of this construct as a vaccine antigen.

There is still an urgent need in the art for better and more effective vaccines against paramyxoviruses, especially RSV. The present invention is directed to this and other unmet needs in the art.

### SUMMARY OF THE INVENTION

In one aspect, the invention provides engineered immunogenic proteins that are derived or modified from the fusion (F) protein of a paramyxovirus (e.g., RSV). They contain an altered soluble F sequence that has one or more modifications relative to a wildtype soluble F sequence of the paramyxovirus. Typically, the engineered soluble F proteins of the invention contain (1) substitutions of two or more negatively charged residues around the β23 strand (D486-A490) with polar or hydrophobic residues, (2) deletion of the P27 peptide (E110-R136), and (3) an engineered intra-protomer disulfide bond that is within the F1 subunit or links the F2 and F1 subunits. Unless otherwise noted, the amino acid numbering of the various sequence modifications described herein is based on human RSV A2 strain F protein (UniProt ID P03420). Some of the engineered soluble F proteins are derived from RSV. In some of the engineered soluble RSV F proteins, the two or more negatively charged residues around the β23 strand are D486 and E487. In some of these embodiments, the substitutions around the β23 strand contain D486N/E487Q or D486L/E487L. In some engineered soluble F proteins of the invention, the engineered disulfide bond is S155C/S290C, S62C/K196C, or E60C/K196C.

In addition to modifications (1)-(3) noted above, some engineered soluble RSV F proteins of the invention can further contain a linker moiety that replaces (1) the furin cleavage site(s) or (2) the unstructured C-terminus of F2 (Q98-R109) and part of the N-terminus of the fusion peptide (FP) (F137-V157). In some of these embodiments, the replaced C-terminus of F2 contains residues N104-R109 (¹⁰⁴NNRARR¹⁰⁹; SEQ ID NO: 31). In some of these embodiments, the replaced part of the N-terminus of the fusion peptide (FP) comprises F137-S146. Some engineered soluble F proteins of the invention further contain substitution of residue S215. In some of these embodiments, residue S215 is replaced with P. Some engineered soluble F proteins of the invention further contain substitution of residue E92. In some of these embodiments, residue E92 is replaced with D, Q, another short and polar residue, or a hydrophobic residue. Some engineered soluble F proteins of the invention can further contain a V185P substitution. Some engineered soluble F proteins of the invention can further contain S46G, K462Q, or both substitutions. Some engineered soluble F proteins of the invention can further contain an engineered intra-protomer disulfide bond S180C/S186C or A177C/T189C in the β3/β4 hairpin. Some engineered soluble F proteins of the invention can further contain an engineered inter-protomer disulfide bond A149C/Y458C. In various embodiments, the engineered soluble RSV F protein of the invention can have an amino acid sequence set forth in any one of SEQ ID NOs: 17-23, a conservatively modified variant thereof, or a substantially identical sequence thereof. In some embodiments, the engineered soluble F protein of the invention can further contain a N-terminal leader sequence. In some embodiments, the engineered soluble F protein of the invention can further contain a C-terminal foldon motif.

In a related aspect, the invention provides nanoparticle vaccines that contain an engineered soluble F protein described herein that is displayed on the surface of a self-assembling nanoparticle. In some of these embodiments, the self-assembling nanoparticle comprises a trimeric sequence, and the C-terminus of the engineered soluble F protein is fused to the N-terminus of the subunit sequence of the nanoparticle. In some embodiments, the employed self-assembling nanoparticle in the nanoparticle vaccine of the invention is a I3-01 variant. In some of these embodiments, the subunit sequence of the I3-01 variant contains SEQ ID NO:25 (I3-01v9b) or SEQ ID NO:26 (I3-01v9c).

In another aspect, the invention provides polynucleotide sequences that encode an engineered soluble F protein or the nanoparticle vaccine described herein. In another aspect, the invention provides pharmaceutical compositions that contain the nanoparticle vaccine or the polynucleotide sequence described herein, plus a pharmaceutically acceptable carrier. In still another aspect, the invention provides methods for preventing or treating a paramyxovirus infection in a subject. These methods entail administering to the subject a therapeutically effective amount of the pharmaceutical composition described herein. Some of these therapeutic methods are directed to treating or preventing RSV infection.

In still another aspect, the invention provides a different class of engineered or redesigned immunogen polypeptides that are derived or modified from the fusion protein (F) of a paramyxovirus. These redesigned soluble F immunogens also contain an altered soluble F sequence that has one or more modifications relative to a wildtype soluble F sequence of the paramyxovirus. In some of these embodiments, the modifications include an intra-protomer engineered disulfide bond that links a pair of β-sheet-forming amino acids in the β3/β4 hairpin or equivalent hairpin in the F1 subunit of the soluble F sequence. Unless otherwise noted, numbering of the hairpin in the immunogens is based on respiratory syncytial virus (RSV).

Some of these different class of engineered soluble F immunogens are derived from the wildtype F sequence of a human RSV. In these embodiments, the engineered disulfide bond is introduced between substituted residues S180C/S186C or A177C/T189C in the β3/β4 hairpin. The amino acid numbering in these embodiments is based on human RSV strain A2 having UniProt ID P03420. In some of these embodiments, the employed wildtype soluble F sequence is shown in SEQ ID NO:1, or a conservatively modified variant or substantially identical sequence thereof. In some embodiments, the modifications relative to the wildtype soluble F sequence also include a mutation in the unstructured C-terminus of the F2 subunit. For example, the redesigned RSV soluble F immunogens can contain truncation of residues 104-109 (NNRARR) (SEQ ID NO:31) and/or P102A substitution in the unstructured F2 C-terminus. In some embodiments, the modifications relative to the wildtype soluble F sequence also include (1) substituting a (GS)n linker sequence for the processed active peptide (P27) (residues E110-R136 and the N-terminus (residues F137-S146) of the fusion peptide, wherein n is any integer from 1 to 5, and/or (2) amino acid substitutions 1379V and M447V. In some exemplified embodiments, the redesigned RSV soluble F immunogens have the amino acid sequence shown in any one of SEQ ID NOs: 36-43 or a conservatively modified variant thereof.

Some of these different class of engineered soluble F immunogens are derived from the wildtype F sequence of a human metapneumovirus (hMPV). In some of these embodiments, the engineered disulfide bond is introduced between substituted residues A147C/A159C in the β3/β4 hairpin. The amino acid numbering in these embodiments is based on hMPV strain CAN97-83 having UniProt ID Q6WB98. In some of these embodiments, the employed wildtype soluble F sequence is shown in SEQ ID NO:44 or SEQ ID NO:45, or a conservatively modified variant or substantially identical sequence thereof. In some embodiments, the modifications relative to the wildtype soluble F sequence also include a mutation in the unstructured C-terminus of the F2 subunit. In some of these embodiments, the mutation in the unstructured F2 C-terminus is replacing the unstructured F2 C-terminus DQLAREEQIENP (SEQ ID NO: 60) and the cleavage site RQSR (SEQ ID NO:49) with a (GS)n linker sequence, wherein n is any integer from 1 to 5. In some exemplified embodiments, the redesigned hMPV soluble F immunogens have the amino acid sequence shown in SEQ ID NO:46 or SEQ ID NO:47, or a conservatively modified variant thereof.

Some of these different class of engineered soluble F immunogens are derived from the soluble F sequence of a parainfluenza virus (hPIV). In some of these embodiments, the engineered disulfide bond is introduced between substituted residues Q159C/A171C in the β1/β2 hairpin. The amino acid numbering in these embodiments is based on recombinant hPIV3/hPIV1 virus having UniProt ID 055888. In some of these embodiments, the employed wildtype soluble F sequence is shown in SEQ ID NO: 48, or a conservatively modified variant or substantially identical sequence thereof. In some embodiments, the modifications relative to the wildtype soluble F sequence also include a mutation in the C-terminus of the F2 subunit. In some of these embodiments, the mutation in the F2 C-terminus is replacing the C-terminus sequence NQESNENTDP (SEQ ID NO:50) and the cleavage site RTER (SEQ ID NO:51) with a (GS)n linker sequence, wherein n is any integer from 1 to 6. In some exemplified embodiments, the redesigned hPIV soluble F immunogens have the amino acid sequence shown in SEQ ID NO:61 or SEQ ID NO:62, or a conservatively modified variant thereof.

In another aspect, the invention provides engineered or redesigned immunogen polypeptides that are derived from the fusion protein (F) of a human respiratory syncytial virus (hRSV). In some embodiments, the redesigned hRSV immunogens contain a modified RSV soluble F sequence that, relative to a wildtype hRSV soluble F sequence, is altered by at least one of the following mutations: (1) deletion of the P27 peptide (residues E110-R136), (2) a modification in the unstructured C-terminus of the F2 subunit (residues Q98-R109), and (3) a truncation of the N-terminus of the fusion peptide (residues F137-V157). In these embodiments, the amino acid numbering is based on human RSV strain A2 having UniProt ID P03420. In some of these embodiments, the employed wildtype RSV soluble F sequence is shown in SEQ ID NO:1, or a conservatively modified variant or substantially identical sequence thereof. In some of these embodiments, the modification in the unstructured F2 C-terminus is (1) truncation of residues 104-109 (NNRARR) (SEQ ID NO:31) and/or (2) P102A substitution. In some embodiments, the truncation of the N-terminus of the fusion peptide is deletion of residues F137-S146.

In some embodiments, modifications in the redesigned RSV soluble F immunogens, relative to the wildtype soluble F sequence, can additionally contain (1) a (GS)n linker between F2 and F1 subunits in the altered soluble RSV sequence, wherein n is any integer from 1 to 6, and/or (2) at least one substitutions selected from the group consisting of 1379V and M447V. In some of these embodiments, the linker contains a sequence GSGS (SEQ ID NO:27) or GSGSGSGS (SEQ ID NO:28). In some exemplified embodiments, the redesigned hRSV soluble F immunogens have the amino acid sequence shown in SEQ ID NO:34 or SEQ ID NO:35, or a conservatively modified variant thereof.

In some embodiments, modifications in the redesigned RSV soluble F immunogens, relative to the wildtype soluble F sequence, can additionally contain an engineered disulfide bond that links a pair of β-sheet-forming amino acids in the β3/β4 hairpin in the F1 subunit. In some of these embodiments, the engineered disulfide bond is introduced between substituted residues S180C/S186C or A177C/T189C. Some specific examples of these redesigned hRSV soluble F immunogens have the amino acid sequence shown in any one of SEQ ID NOs: 36, 38, 40 and 42, or a conservatively modified variant thereof.

In some embodiments, the modifications in the redesigned RSV soluble F immunogens, relative to the wildtype soluble F sequence, can additionally contain substitutions of one or two amino acid residues between β strands β3 and β4. In some of these embodiments, the amino acid substitutions are S182G and N183P. Some specific examples of these redesigned hRSV soluble F immunogens have the amino acid sequence shown in any one of SEQ ID NOs: 37, 39, 41 and 43, or a conservatively modified variant thereof.

In addition to the various sequence modifications or mutations noted above, some redesigned soluble F immunogens of the invention can additionally include a trimerization motif at the C-terminus. In some of these embodiments, the employed trimerization motif is foldon or viral capsid protein SHP.

In another aspect, the invention provides paramyxovirus vaccine compositions that contain a redesigned soluble F immunogen described herein that is displayed on the surface of a self-assembling nanoparticle. In some embodiments, the self-assembling nanoparticle contains a trimeric sequence, and the C-terminus of the immunogen polypeptide is fused to N-terminus of the subunit sequence of the nanoparticle. In another aspect, the invention provides pharmaceutical compositions that contain a redesigned soluble F immunogen or a nanoparticle vaccine described herein, and a pharmaceutically acceptable carrier. In another aspect, the invention provides polynucleotide sequences that encode a redesigned soluble F immunogen described herein or a subunit sequence of the vaccine compositions displaying a redesigned soluble F immunogen described herein.

A further understanding of the nature and advantages of the present invention may be realized by reference to the remaining portions of the specification and claims.

### DESCRIPTION OF THE DRAWINGS

FIG. 1. Analysis of sources of RSV F metastability. (A) Amino-acid sequence and secondary structure alignment of RSV prefusion and postfusion F, SEQ ID NO: 63 (from Fig. S3, McLellan et al., Science 2013, 340:1113-1117). Two potential sources of metastability, the β3/β4 hairpin and the β23 strand, are circled with dotted-line boxes. (B) The β3/β4 hairpin (SEQ ID NO:53). Left: prefusion and postfusion F; Right: zoomed-in views of β3/β4 in pre- and post-fusion states (potential sites of mutation are labeled). (C) The β23 strand (SEQ ID NO:64). Left: side view of β23 in the prefusion F trimer structure; Right: top view of β23 in the prefusion F trimer structure (top) and a zoomed-in view of β23 around the 3-fold axis (bottom).

FIG. 2. Rational design of I3-01v9b/c to achieve the optimal display of trimeric antigens on the nanoparticle surface. (A) Structural model of I3-01v9a (SEQ ID NO: 24), which has an extended N-terminal helix. (B) Scheme of the process used to design I3-01v9b/c (SEQ ID NO:25). (C) The nsEM analysis of EBOV GP-I3-01v9b trimer. Top: 2D classes; Bottom: side view and top view of the 3D model.

FIG. 3. Design and negative-stain EM analysis of RSV prefusion F trimer-presenting nanoparticles. (A) Structural modeling of RSV prefusion F trimers on three 1c-SApNPs including ferritin 24-mer and two 60-mers, E2p and I3-01v9b. (B) EM analysis of DS-Cav1 on three 1c-SApNPs. (C) EM analysis of sc9-10 DS-Cav1 on three 1c-SApNPs. (D) V2-Ext-P2DB6-D-L2 and NQ on FR and NQ on I3-01v9b 1c-SApNPs. (E) V2-Ext-P2DB6-GDQ-L2 and NQ on FR and NQ on I3-01v9b 1c-SApNPs. For (D) and (E), zoomed-in view of F-FR nanoparticles are shown, with closed pre-F trimers on the surface.

FIG. 4. Negative-stain EM of RSV prefusion F trimers on nanoparticle platforms. (A) EM images of DS-Cav1, sc9-10 DS-cav1, and SC-TM RSV F displayed on ferritin nanoparticles. DS-Cav1-FR5 failed to form nanoparticles properly. sc9-10-DS-Cav-FR5 form nanoparticles but the F trimers appeared to be open on the ferritin particle surface (one such particle is highlighted in the red box). SC-TM-FR failed to form nanoparticles properly. (B) EM images of two major F trimer designs displayed on ferritin and E2p particles. Column 1: V2-Ext-SSGP on ferritin with a 10GS linker and purified by D25 and MPE8. Column 2: V2-Ext-AT on ferritin with a 5GS linker and purified by D25 and MPE8. Column 3: V2-Ext-AT on E2p 60-mer with locking domain (LD4) and with LD4 and a T-cell epitope PADRE. All newly designed F trimers have well-formed, closed prefusion conformation on the nanoparticle surface (one such V2-Ext-AT-FR5 particle is highlighted).

### DETAILED DESCRIPTION

### I Overview

As a class I viral fusion protein, RSV F has inherent metastability, which can manifest in different forms compared with other class I fusion proteins such as HIV-1 envelope glycoprotein (Env). First, the prefusion RSV F is very unstable and prone to change its conformation to the postfusion state, a phenomenon that has been extensively studied in the field. Second, the RSV prefusion F trimer is prone to dissociate into monomers or become open trimers, unless it is locked by an inter-protomer disulfide bond, which will result in an adverse effect on the multivalent display of prefusion F trimers on nanoparticles through the gene fusion approach.

The present invention is derived in part from studies undertaken by the inventors to rationally design new, stable RSV prefusion F trimers by minimizing F metastability. The inventors first examined the expression, purification, and structure of DS-Cav1, SC-TM, and sc9-10 DS-Cav1, three known prefusion RSV F design. It was observed that DS-Cav1 and SC-TM produced a lot of aggregates and non-trimer F species, whereas sc9-10 DS-Cav1 showed a single trimer peak with high yield and high purity. In the negative-stain EM analysis, DS-Cav1 and SC-TM were found to be monomers and a monomer/trimer mix, respectively, whereas sc9-10 DS-Cav1 appeared to be high-purity closed prefusion F trimers. When displayed on protein nanoparticles (NPs), all three prefusion F designs showed poor performance with low yield and low purity. At the core of the invention, the inventors explored novel mutations that could substantially reduce metastability of prefusion F conformation, e.g., an RSV F mutant protein in prefusion conformation achieved by an engineered disulfide bond (e.g., S155C/S290C exemplified herein). To this end, the inventors discovered that mutations of a pair of negatively charged residues in the β23 strand (D486-A490), e.g., D486 and E487, can greatly stabilize the prefusion F in a closed trimer conformation.

The inventors additionally examined other mutations in the “base” prefusion F structure, into which the noted mutations in the β23 strand are to be introduced, that may further improve the antigenic profile of the engineered F protein. Other than the intra-protomer disulfide bond in the β23 strand than locks the RSV F protein in a prefusion conformation, these additional mutations include a cleavage site linker that replaces the unstructured F2 C terminus, P27 peptide and fusion peptide, a S215P mutation, and an E92D mutation. This F construct is termed “V2-Ext-PDB6-D”. It was found that a construct containing this set of mutations, V2-Ext-PDB6-D, produced high yield and high purity prefusion F with a small fraction of closed trimers. Using this prefusion F base design, the inventors further examined a V185P mutation in the β3/β4 hairpin (K176-S190) and two types of mutation to a pair of negatively charged residues D486 and E487 in the β23 strand (D486-A490). Three β23 strands form repulse interactions around the 3-fold axis directly above the α10 coiled-coil. While the V185P mutation may destabilize the postfusion F structure, the inventors hypothesized that the D486-E487 pair causes prefusion F trimer to open to facilitate cell entry and thus polar or hydrophobic mutations to these two residues can stabilize prefusion F in a closed trimer conformation. Indeed, negative-stain EM analysis of various constructs proved the hypothesis. Then, the inventors included two mutations, S46G and K465Q, into the 1^{st} base design to improve prefusion F folding. This 2^{nd} base design is termed “V2-Ext-PDB6-GDQ” and used to test the V185P mutation and mutations to the D486-E487 pair. While S46G and K462Q did improve prefusion F folding, they also reduced the ratio of closed prefusion F trimers. Both V2-Ext-PDB6-D and V2-Ext-PDB6-GDQ derivatives can be developed as soluble trimer vaccines or displayed on single-component self-assembling protein nanoparticles (1c-SApNPs) as virus-like particle (VLP)-type vaccines. Because the F proteins of other Paramyxoviruses (e.g., hMPV and PIV) are structurally similar to RSV F, the same design strategy can be applied to the other members of the Paramyxoviridae family.

In additional studies, the inventors explored a different set of minimum mutations that can be introduced into paramyxovirus fusion (F) glycoproteins to generate stabilized immunogens. As detailed herein, these paramyxovirus F protein trimer immunogens are resigned by introducing structural modifications in a soluble F sequence that can stabilize the F trimer in a prefusion state. Some of the redesigned soluble F immunogens are stabilized by introducing an engineered disulfide bond in a β hairpin in the F1 subunit. Some of the redesigned soluble F immunogens are stabilized by introducing other novel mutations in the soluble F sequence. The inventors further displayed the redesigned F trimer immunogen on self-assembling nanoparticles as VLP-type vaccines, and developed methods for industrial production and tag-free antigen-specific purification of the immunogens. Because hMPV and PIV F proteins are structurally similar to RSV F, the same stabilizing design validated for RSV F was also utilized for redesigning hMPV and PIV3 vaccine immunogens. These studies provide a universal design strategy for paramyxovirus prefusion F-based vaccine design.

The invention accordingly provides paramyxovirus immunogens and vaccine compositions in accordance with the design strategy described herein. Related polynucleotide sequences, expression vectors and pharmaceutical compositions are also provided in the invention. Unless otherwise specified herein, the vaccine immunogens of the invention, the encoding polynucleotides, expression vectors and host cells, as well as the related therapeutic applications, can all be generated or performed in accordance with the procedures exemplified herein or routinely practiced methods well known in the art. See, e.g., Methods in Enzymology, Volume 289: Solid-Phase Peptide Synthesis, J. N. Abelson, M. I. Simon, G. B. Fields (Editors), Academic Press; 1st edition (1997) (ISBN-13:978-0121821906); U.S. Pat. Nos. 4,965,343, and 5,849,954; Sambrook et al., Molecular Cloning: A Laboratory Manual, Cold Spring Harbor Press, N.Y., (3^{rd} ed., 2000); Brent et al., Current Protocols in Molecular Biology, John Wiley & Sons, Inc. (ringbou ed., 2003); Davis et al., Basic Methods in Molecular Biology, Elsevier Science Publishing, Inc., New York, USA (1986); or Methods in Enzymology: Guide to Molecular Cloning Techniques Vol. 152, S. L. Berger and A. R. Kimmerl Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998). The following sections provide additional guidance for practicing the compositions and methods of the present invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Oxford Dictionary of Biochemistry and Molecular Biology, Smith et al. (Eds.), Oxford University Press (revised ed., 2000); Encyclopaedic Dictionary of Chemistry, Kumar (Ed.), Anmol Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). Further clarifications of some of these terms as they apply specifically to this invention are provided herein.

As used herein, the singular forms “a,” “an,” and “the,” refer to both the singular as well as plural, unless the context clearly indicates otherwise. For example, “an Env-derived trimer” can refer to both single or plural Env-derived trimer molecules, and can be considered equivalent to the phrase “at least one Env-derived trimer.”

As used herein, the terms “antigen” or “immunogen” are used interchangeably to refer to a substance, typically a protein, which is capable of inducing an immune response in a subject. The term also refers to proteins that are immunologically active in the sense that once administered to a subject (either directly or by administering to the subject a nucleotide sequence or vector that encodes the protein) is able to evoke an immune response of the humoral and/or cellular type directed against that protein. Unless otherwise noted, the term “vaccine immunogen” is used interchangeably with “protein antigen” or “immunogen polypeptide”.

The term “conservatively modified variant” applies to both amino acid and nucleic acid sequences. With respect to particular nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For polypeptide sequences, “conservatively modified variants” refer to a variant which has conservative amino acid substitutions, amino acid residues replaced with other amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Epitope refers to an antigenic determinant. These are particular chemical groups or peptide sequences on a molecule that are antigenic, such that they elicit a specific immune response, for example, an epitope is the region of an antigen to which B and/or T cells respond. Epitopes can be formed both from contiguous amino acids or noncontiguous amino acids juxtaposed by tertiary folding of a protein.

Effective amount of a vaccine or other agent that is sufficient to generate a desired response, such as reduce or eliminate a sign or symptom of a condition or disease, such as bronchiolitis or pneumonia. For instance, this can be the amount necessary to inhibit viral replication or to measurably alter outward symptoms of the viral infection. In general, this amount will be sufficient to measurably inhibit virus (for example, hRSV) replication or infectivity. When administered to a subject, a dosage will generally be used that will achieve target tissue concentrations that has been shown to achieve in vitro inhibition of viral replication. In some embodiments, an “effective amount” is one that treats (including prophylaxis) one or more symptoms and/or underlying causes of any of a disorder or disease, for example to treat RSV infection. In some embodiments, an effective amount is a therapeutically effective amount. In some embodiments, an effective amount is an amount that prevents one or more signs or symptoms of a particular disease or condition from developing, such as one or more signs or symptoms associated with bronchiolitis.

Unless otherwise noted, a fusion protein is a recombinant protein containing amino acid sequence from at least two unrelated proteins that have been joined together, via a peptide bond, to make a single protein. Thus, it does not encompass the naturally existing paramyxoviruses surface antigen that is termed fusion (F) protein as described herein. The unrelated amino acid sequences can be joined directly to each other or they can be joined using a linker sequence. As used herein, proteins are unrelated, if their amino acid sequences are not normally found joined together via a peptide bond in their natural environment(s) (e.g., inside a cell). For example, the amino acid sequences of bacterial enzymes such as B. stearothermophilus dihydrolipoyl acyltransferase (E2p) and the amino acid sequences of a soluble paramyxovirus F glycoproteins are not normally found joined together via a peptide bond.

Immunogen is a protein or a portion thereof that is capable of inducing an immune response in a mammal, such as a mammal infected or at risk of infection with a pathogen. Administration of an immunogen can lead to protective immunity and/or proactive immunity against a pathogen of interest.

Immunogenic composition refers to a composition comprising an immunogenic polypeptide that induces a measurable CTL response against virus expressing the immunogenic polypeptide, or induces a measurable B cell response (such as production of antibodies) against the immunogenic polypeptide.

Sequence identity or similarity between two or more nucleic acid sequences, or two or more amino acid sequences, is expressed in terms of the identity or similarity between the sequences. Sequence identity can be measured in terms of percentage identity; the higher the percentage, the more identical the sequences are. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

Homologs or orthologs of nucleic acid or amino acid sequences possess a relatively high degree of sequence identity/similarity when aligned using standard methods. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The term “subject” refers to any animal classified as a mammal, e.g., human and non-human mammals. Examples of non-human animals include dogs, cats, cattle, horses, sheep, pigs, goats, rabbits, and etc. Unless otherwise noted, the terms “patient” or “subject” are used herein interchangeably. Preferably, the subject is human.

The term “treating” or “alleviating” includes the administration of compounds or agents to a subject to prevent or delay the onset of the symptoms, complications, or biochemical indicia of a disease (e.g., an hRSV infection), alleviating the symptoms or arresting or inhibiting further development of the disease, condition, or disorder. Subjects in need of treatment include those already suffering from the disease or disorder as well as those being at risk of developing the disorder. Treatment may be prophylactic (to prevent or delay the onset of the disease, or to prevent the manifestation of clinical or subclinical symptoms thereof) or therapeutic suppression or alleviation of symptoms after the manifestation of the disease.

Vaccine refers to a pharmaceutical composition that elicits a prophylactic or therapeutic immune response in a subject. In some cases, the immune response is a protective immune response. Typically, a vaccine elicits an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents. In some embodiments of the invention, vaccines or vaccine immunogens or vaccine compositions are expressed from fusion constructs and self-assemble into nanoparticles displaying an immunogen polypeptide or protein on the surface.

Virus-like particle (VLP) refers to a non-replicating, viral shell, derived from any of several viruses. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. Methods for producing particular VLPs are known in the art. The presence of VLPs following recombinant expression of viral proteins can be detected using conventional techniques known in the art, such as by electron microscopy, biophysical characterization, and the like. See, for example, Baker et al. (1991) Biophys. J. 60:1445-1456; and Hagensee et al. (1994) J. Virol. 68:4503-4505. For example, VLPs can be isolated by density gradient centrifugation and/or identified by characteristic density banding. Alternatively, cryoelectron microscopy can be performed on vitrified aqueous samples of the VLP preparation in question, and images recorded under appropriate exposure conditions.

A self-assembling nanoparticle refers to a ball-shape protein shell with a diameter of tens of nanometers and well-defined surface geometry that is formed by identical copies of a non-viral protein capable of automatically assembling into a nanoparticle with a similar appearance to VLPs. A notable example of self-assembling nanoparticles is engineered protein I3-01 (Hsia et al., Nature 535, 136-139, 2016) and variants derived therefrom, including I3-01v9b and I3-01v9c exemplified herein. Other examples include ferritin (FR), which is conserved across species and forms a 24-mer, as well as B. stearothermophilus dihydrolipoyl acyltransferase (E2p), Aquifex aeolicus lumazine synthase (LS), and Thermotoga maritima encapsulin, which all form 60-mers. Self-assembling nanoparticles can form spontaneously upon recombinant expression of the protein in an appropriate expression system. Methods for nanoparticle production, detection, and characterization can be conducted using the same techniques developed for VLPs.

### III. Paramyxoviruses and the Fusion (F) Glycoproteins

The invention provides novel engineered immunogenic proteins and vaccine compositions that contain a modified soluble F glycoprotein sequence of Paramyxoviruses. Paramyxovirus F proteins are homotrimeric. They have a hydrophobic fusion peptide (FP), two heptad repeat regions (HRA and HRB), are anchored at the surface by a single-pass transmembrane domain (TM), and contain a c-terminal cytoplasmic tail. Using RSV for illustration, the F gene of paramyxovirus encodes a type I integral membrane protein that is synthesized as a 574 amino acid inactive precursor, F₀. Three F₀ monomers assemble into a trimer and, as the trimer passes through the Golgi, the monomers are activated by a furin-like host protease. The protease cleaves twice, after amino acids 109 and 136, generating three polypeptides. The N-terminal and C-terminal cleavage products are the F₂ and F₁ subunits (named in order of size), respectively, and are covalently linked to each other by two disulfide bonds. The intervening 27 amino acid peptide, P27, contains 2 or 3 N-linked glycans, but dissociates after cleavage. The F₂ subunit contains two N-linked glycans, whereas the larger F₁ subunit contains a single N-linked glycosylation site. Unlike the others, this F₁ glycan is essential for the protein to cause membrane fusion.

In general art, a wildtype soluble F sequence of a paramyxovirus refers to the entire ectodomain of the fusion glycoprotein (F) of the paramyxovirus. Using RSV F protein as an example, the soluble F sequence (amino acids 1-529) typically contains from its N-terminus to C-terminus: the leader sequence, followed by the F2 subunit, the processed active peptide (P27) peptide, and the ectodomain portion of the F1 subunit which includes at its N-terminus the fusion peptide (FP). A deletion of the wildtype soluble F sequence at the C-terminus results in an F construct termed Fd (amino acids 1-513), which has been used in structure determination of RSV F protein in both prefusion and postfusion conformations. Therefore, the term wildtype soluble F as used herein can refer to Fd, and in some embodiments can also be extended by adding more amino acids at the C-terminus until it contains the full-length ectodomain portion of the F1 subunit. Unless otherwise noted, amino acid numbering of the various components of an RSV soluble F sequence is based on human RSV A2 strain with accession no. P03420 (McLellan et al., J. Virol. 85:7788-96, 2011). In some embodiments, the wildtype soluble RSV F sequence from which the engineered soluble F immunogens of the invention are derived is shown in SEQ ID NO:1. Similarly, amino acid numbering in the soluble F sequences of the other paramyxoviruses are also based on specific viral strains and/or secondary structures described herein.

hRSV A2 strain “wildtype” soluble F sequence (SEQ ID NO: 1):
MELLILKANAITTILTAVTFCFASGQNITEEFYQSTCSAVSKGYLSALRTGWYTS
VITIELSNIKENKCNGTDAKVKLIKQELDKYKNAVTELQLLMQSTPPTNNRARR
ELPRFMNYTLNNAKKTNVTLSKKRKRRFLGFLLGVGSAIASGVAVSKVLHLE
GEVNKIKSALLSTNKAVVSLSNGVSVLTSKVLDLKNYIDKQLLPIVNKQSCSIS
NIETVIEFQQKNNRLLEITREFSVNAGVTTPVSTYMLTNSELLSLINDMPITNDQ
KKLMSNNVQIVRQQSYSIMSIIKEEVLAYVVQLPLYGVIDTPCWKLHTSPLCTT
NTKEGSNICLTRTDRGWYCDNAGSVSFFPQAETCKVQSNRVFCDTMNSLTLPS
EINLCNVDIFNPKYDCKIMTSKTDVSSSVITSLGAIVSCYGKTKCTASNKNRGII
KTFSNGCDYVSNKGMDTVSVGNTLYYVNKQEGKSLYVKGEPIINFYDPLVFPS
DEFDASISQVNEKINQSLAFIRKSDELL

As used herein, unstructured F2 C-terminus of a paramyxovirus F glycoprotein refers to segment of the amino acid sequence at the C-terminus of its F2 subunit that is flexible and therefore not visible in a three-dimensional structure of the prefusion F protein. Many paramyxoviruses have an unstructured F2 C-terminus. For example, based on the crystal structures that have been determined for various RSV prefusion F constructs, it was found that C-terminus of F2 is always unstructured. A crystal structure solved for an hMPV prefusion F construct indicates that the C-terminus of F2 of the hMPV is unstructured. Similarly, an EM structure has been solved for a PIV3 prefusion F construct, which shows that the C-terminus of F2 of the PIV3 is unstructured.

### IV. Engineered Paramyxovirus Soluble F Immunogens

The invention provides engineered (redesigned or modified) soluble F sequences of paramyxoviruses that can be employed for generating vaccine compositions. The redesigned soluble F trimer immunogens or proteins are stabilized by introducing modifications into the wildtype soluble F sequences of paramyxoviruses. Some specific wildtype soluble F sequences of specific hRSV strains are exemplified herein. Due to functional similarity and sequence homology among different strains of a given paramyxovirus, redesigned soluble F immunogens derived from other known paramyxovirus F protein ortholog sequences can also be generated in accordance with the redesign strategy described herein. There are many known paramyxovirus ortholog or homolog F protein sequences that have been described in the literature. See, e.g., Collins et al., Proc. Natl. Acad. Sci. USA 81:7683-7, 1984; Hause et al., PLOS ONE 12: e0175792, 2017; Chang et al., Viruses 4:613-636, 2012; and Amanda et al., J. Virol. 81: 8303-8314, 2007. As detailed herein, the engineered soluble F proteins of the invention contain one or more specific stabilizing mutations in the corresponding wildtype soluble F sequences. These mutations include (a) substitutions of two or more negatively charged residues around the β23 strand as exemplified herein for RSV, (b) deletion of the P27 peptide, (c) an engineered intra-protomer disulfide bond that is either within the F1 subunit or links the F2 and F1 subunits, and (d) an engineered inter-protomer disulfide bond as exemplified herein for RSV. In some embodiments, the engineered soluble F proteins of the invention can contain a combination of any 2 (e.g., a and d) or 3 (e.g., a, c and d) of these mutations. In some embodiments, the engineered soluble F proteins can contain all of these 4 mutations.

In some embodiments, relative to their wildtype counterpart sequences, the engineered soluble F proteins contain substitution of two or more negatively charged residues around the β23 strand with polar or hydrophobic residues. As used herein, the negatively charged residues around the β23 strand refer to a negatively charged stretch that is centered at or around β23. It can include the β23 strand plus 1-2 residues up- and downstream. Other than these substitutions, the engineered soluble F sequences of the invention typically also have a deletion of the processed active peptide (P27), and/or contain an engineered and stabilizing disulfide bond which is present within the Fs subunit or links the F2 and F1 subunits, and locks the protein in a prefusion conformation. Using the prototype human RSV strain A2 F protein (UniProt ID P03420) as reference, the P27 peptide corresponds to residues E110-R136, and the negatively charged stretch encompasses residues at and around the β23 strand D486-A490. Of note, residue 485 is a Ser(S) in human RSV but the equivalent residue in human MPV is a Glu (E453). This residue is also included as part of the negatively charged stretch around β23, that forms repulsive interactions around the trimer axis. In some embodiments, the engineered soluble F proteins of the invention have two residues in the β23 strand, D486 and E487, replaced with polar or hydrophobic residues. In some embodiments for other paramyxoviruses such as hMPV, the immediate upstream residue (E453) of the β23 strand can also be replaced with polar or hydrophobic residues. In some embodiments, the engineered disulfide bond is S155C/S290C, which is present within the F1 subunit. In some other embodiments, the engineered disulfide bond is S62C/K196C or E60C/K196C, which links the F2 and F1 subunits.

Other than the above-noted modifications, some engineered soluble F proteins of the invention can contain one or more additional mutations in comparison to their wildtype counterpart sequences. In some embodiments, they have insertion of a linker moiety that substitute for the furin cleavage sites. In some other embodiments, a linker moiety is inserted to replace the unstructured C-terminus of F2 and part of the N-terminus of the fusion peptide (FP). Again, using human RSV strain A2 as reference, the replaced C-terminus of F2 corresponds to residues N104-R109 (NNRARR; SEQ ID NO: 31). The replaced N-terminus of the fusion peptide (FP) (F137-V157) contains residues F137-S146. In some preferred embodiments, the substituting linker moiety is a GS rich linker, e.g., (GS)n wherein n can be any integer from 1 to about 5. In various embodiments, the linker contains the sequence GSGS (SEQ ID NO:27) or GSGSGSGS (SEQ ID NO:28).

In some embodiments, the engineered soluble F proteins of the invention can additionally contain substitution of a residue corresponding to S215 in the F glycoprotein of RSV strain A2. In some embodiments, the engineered soluble F proteins of the invention can additionally contain substitution of a residue corresponding to E92 in the F glycoprotein of RSV strain A2. For example, residue E92 in the soluble F protein can be replaced with D, Q, or another short, polar residue, or in some cases a hydrophobic residue, e.g., L and I, to form hydrophobic interactions with the neighboring protomers. In some embodiments, the engineered soluble F proteins of the invention can additionally contain substitutions at residues S46 and K462. In some of these embodiments, the substitutions are S46G/K462Q. In still some other embodiments, the engineered soluble F proteins of the invention contains an engineered disulfide bond that links a pair of β-sheet-forming amino acids in the β3/β4 hairpin or equivalent hairpin in the F1 subunit. As demonstrated herein, this engineered disulfide bond functions to further reduce metastability and increase stability of the prefusion soluble F sequence. It is noted that this β3/β4 hairpin in RSV and MPV has its equivalent in the β1/β2 hairpin in PIVs. In some of these embodiments, this disulfide bond is formed via substitutions A177C/T189C, with amino acid numbering based on human RSV strain A2.

Some specific examples of the engineered RSV soluble F sequences or immunogens are shown in SEQ ID NOs: 17-23. In addition to these exemplified sequences, engineered RSV soluble F immunogens of the invention also include sequences that are conservatively modified variants of these sequences or substantially identical sequences.

Other than the modifications in the wildtype soluble F sequences as described above, some engineered soluble F antigens or immunogenic proteins of the invention can contain a N-terminal leader sequence (or “signal peptide”). In some of these embodiments, the N-terminal leader contains the sequence MELLILKANAITTILTAVTFCFASG (SEQ ID NO:2) as exemplified herein. In some embodiments, the engineered soluble F proteins of the invention can also include one or more C-terminal structural motifs that facilitate trimerization. For example, the engineered proteins can contain a C-terminal foldon motif, GYIPEAPRDGQAYVRKDGEWVLLSTFL (SEQ ID NO:7), as exemplified herein. In some of these embodiments, a restriction site such as “AS” exemplified herein can be appended to the N-terminus of the foldon motif.

### V. Engineered Paramyxovirus Soluble F Trimers with a Different Set of Mutations

In addition to the modifications introduced into soluble F trimers as described above, the invention also provides engineered paramyxovirus soluble F immunogens that contain a different set of mutations relative to the wildtype F sequences. Again, some specific wildtype soluble F sequences of specific hRSV, hMPV and hPIV3 strains are exemplified herein for these additional engineered paramyxovirus soluble F immunogens. Due to functional similarity and sequence homology among different strains of a given paramyxovirus, redesigned soluble F immunogens derived from other known paramyxovirus F protein ortholog sequences can also be generated in accordance with the redesign strategy described herein. There are many known paramyxovirus ortholog or homolog F protein sequences that have been described in the literature. See, e.g., Collins et al., Proc. Natl. Acad. Sci. USA 81:7683-7, 1984; Hause et al., PLOS ONE 12: e0175792, 2017; Chang et al., Viruses 4:613-636, 2012; and Amanda et al., J. Virol. 81:8303-8314, 2007.

As detailed herein (e.g., Examples 8-14), some of these additional redesigned soluble F immunogens of the invention contain an engineered disulfide that links a pair of β-sheet forming amino acid residues in the β3/β4 hairpin (or an equivalent hairpin) in the F1 subunit of the soluble F sequence. Some other redesigned soluble F proteins contain a set of mutations that can stabilize the F trimer in a prefusion state. These include mutations in the F2 C-terminus, deletion of the P27 peptide, and mutations in the N-terminus of the fusion peptide in the F1 subunit. Some other redesigned soluble F proteins can contain the engineered disulfide bond, as well as one or more of these mutations.

In one aspect, the invention provides engineered or redesigned immunogen proteins or polypeptides that are derived from the fusion glycoprotein (F) of any paramyxovirus. These immunogens contain an altered soluble F sequence that has modifications relative to wildtype soluble F sequence of the paramyxovirus. The modifications include an engineered disulfide bond that links a pair of β-sheet-forming amino acids in the β3/β4 hairpin or equivalent hairpin in the F1 subunit of the paramyxovirus F protein. This hairpin is the β3/β4 hairpin in RSV and MPV. In PIVs, the equivalent hairpin is the β1/β2 hairpin. Some of these immunogens are derived from the wildtype soluble F sequence of RSV, e.g., human RSV (hRSV). In some of these embodiments, the engineered disulfide bond is generated by amino acid substitution S180C/S186C in the β3/β4 hairpin. In some other embodiments, the engineered disulfide bond is generated by amino acid substitution A177C/T189C in the hairpin. The amino acid number in the redesigned RSV F immunogens of the invention is based on F glycoprotein sequence of human RSV strain A2, which has a UniProt ID number P03420. In some embodiments, the wildtype soluble RSV F sequence from which the redesigned immunogens are derived is shown in SEQ ID NO:1.

In some redesigned RSV soluble F immunogens of the invention, other than the engineered disulfide bond, modifications of the wildtype sequence also include a mutation in the unstructured C-terminus of the F2 subunit. For example, the redesigned soluble F sequence can include a truncation in the unstructured F2 C-terminus. As specific exemplification, residues 104-109 (NNRARR) (SEQ ID NO:31) in the F2 C-terminus can be deleted. Additionally, or alternatively, an amino acid substitution in the unstructured F2 C-terminus can also be introduced into the redesigned soluble F sequence. For example, some of the redesigned RSV soluble F immunogens of the invention can contain a P102A substitution in the F2 C-terminus.

In some redesigned RSV soluble F immunogens of the invention, modifications of the wildtype sequence can contain one or more other mutations. These include, e.g., substitution of short GS linker sequence for the processed active peptide (P27) (residues E110-R136) and the N-terminus (e.g., residues F137-S146) of the fusion peptide. In various embodiments, the GS linker can have a sequence formula (GS)ₙ, wherein n is any integer from 1 to about 5. The additional modifications can also include further substitutions in the F1 subunit. These include, e.g., substitutions I379V and M447V, as exemplified herein using the wildtype soluble F sequence shown in SEQ ID NO:1.

Some specific examples of redesigned RSV soluble F sequences or immunogens are shown in SEQ ID NOs: 36-43. In addition to these exemplified sequences, redesigned RSV soluble F immunogens of the invention also include sequences that are conservatively modified variants of these sequences or substantially identical sequences.

Some of the redesigned soluble F immunogens containing an engineered disulfide bond are derived from the wildtype soluble F sequence of a metapneumovirus, e.g., a hMPV. In some of these embodiments, the engineered disulfide bond is generated by amino acid substitution A147C/A159C in the β3/β4 hairpin. The amino acid numbering is based on the crystal structure (PDB ID: 5WB0) and the UniProt definition for hMPV strain CAN97-83 with the ID Q6WB98. In some embodiments, the wildtype soluble MPV F sequence from which the redesigned immunogens are derived are shown in SEQ ID NO:44 or SEQ ID NO:45. These two sequences are based on hMPV isolate TN03.03.19, which has GenBank ID AEZ52364.

Other than the engineered disulfide bond, modifications of the wildtype sequence in some redesigned hMPV soluble F immunogens of the invention also include a mutation in the unstructured C-terminus of the F2 subunit. In some of these embodiments, the mutation in the unstructured F2 C-terminus is deletion of the unstructured F2 C-terminus DQLAREEQIENP (SEQ ID NO:60) and the cleavage site RQSR (SEQ ID NO:49). Additionally, the deleted sequence can be replaced with a shorter (GS)n linker sequence noted above. Some specific examples of redesigned hMPV soluble F immunogens of the invention are shown in SEQ ID NOs: 46 and 47. In addition to these exemplified sequences, redesigned hMPV soluble F immunogens of the invention also include sequences that are conservatively modified variants of these sequences or substantially identical sequences.

Some other redesigned soluble F immunogens containing an engineered disulfide bond are derived from the wildtype soluble F sequence of human parainfluenza viruses. These include, e.g., human parainfluenza viruses 1-5 (hPIV1-5). Using hPIV3 as exemplification, some of the redesigned hPIV3 soluble immunogens have an engineered disulfide bond that is generated by amino acid substitution Q159C/A171C in the β1/β2 hairpin. The amino acid numbering is based on the cryo-EM structure (PDB ID: 6MJZ) and the UniProt definition for a recombinant PIV3/PIV1 virus with the ID (O55888). In some embodiments, the wildtype soluble MPV F sequence from which the redesigned immunogens are derived are shown in SEQ ID NO: 48. This sequence is from the F protein of hPIV3 strain “HPIV3/USA/629-D01959/2007, which has GenBank ID AGW51052. Due to substantial structural similarity among different PIVs (e.g., hPIV3 and hPIV5 as exemplified herein), the redesign strategy exemplified herein for hPIV3 can be readily applied to the other PIVs.

Other than the engineered disulfide bond, modifications of the wildtype sequence in some redesigned PIV soluble F immunogens of the invention can also include a mutation in the C-terminus of the F2 subunit. In some of these embodiments, the mutation in the F2 C-terminus is deletion of NQESNENTDP (SEQ ID NO:50) and the cleavage site RTER (SEQ ID NO:51). Additionally, the deleted sequence can be replaced with a shorter (GS)n linker sequence noted above. Some specific examples of redesigned hMPV soluble F immunogens of the invention are shown in SEQ ID NOs: 61 and 62. In addition to these exemplified sequences, redesigned PIV soluble F immunogens of the invention also include sequences that are conservatively modified variants of these sequences or substantially identical sequences.

In another aspect, the invention provides engineered or redesigned RSV soluble F immunogens or proteins that are stabilized by a specific set of modifications of a wildtype RSV soluble F sequence. Using amino acid numbering based on human RSV strain A2 (UniProt ID P03420), modifications of the wildtype soluble RSV F sequence in these redesigned immunogens include (1) deletion of the P27 peptide (residues E110-R136), (2) a modification in the unstructured C-terminus of the F2 subunit (residues Q98-R109), and (3) a truncation of the N-terminus of the fusion peptide (e.g., residues F137-V157). In some embodiments, the wildtype soluble RSV F sequence from which the redesigned immunogens are derived are shown in SEQ ID NO: 1.

In some of these immunogen proteins, the modifications in the unstructured F2 C-terminus are truncation of residues 104-109 (NNRARR; SEQ ID NO:31) and P102A substitution. In some embodiments, the truncation of the N-terminus of the fusion peptide is deletion of residues F137-S146. In some embodiments, the redesigned RSV soluble F immunogen polypeptide contains an inserted (GS)n linker between F2 and F1. In the linker formula, n can be any integer from 1 to about 5. In various embodiments, the linker contains the sequence GSGS (SEQ ID NO:27) or GSGSGSGS (SEQ ID NO:28). In some embodiments, the redesigned RSV soluble F immunogen polypeptide contain amino acid substitution 1379V and/or M447V. An exemplary redesigned RSV soluble F immunogen sequence is shown in SEQ ID NO:34 or SEQ ID NO: 35. Other than these exemplified sequences, redesigned RSV soluble F immunogens of the invention also include sequences that are conservatively modified variants of these sequences or substantially identical sequences.

Other than the specific set of mutations noted above, modifications in the redesigned RSV soluble F immunogens of the invention, relative to the wildtype soluble RSV F sequence, can also include an engineered disulfide bond. The engineered disulfide, which links a pair of β-sheet-forming amino acids in the β3/β4 hairpin in the F1 subunit, functions to reduce metastability and increase stability of the prefusion soluble F sequence. In some of these embodiments, the engineered disulfide bond that is generated by amino acid substitutions S180C/S186C in the β3/β4 hairpin. In some other embodiments, the engineered disulfide bond that is generated by amino acid substitutions A177C/T189C in the β3/β4 hairpin. Some examples of these redesigned RSV soluble F immunogen sequences of the invention are shown in SEQ ID NOs: 36, 38, 40 and 42. In addition to these exemplified sequences, redesigned RSV soluble F immunogens of the invention also include sequences that are conservatively modified variants of these sequences or substantially identical sequences.

In some other embodiments, modifications in the redesigned RSV soluble F immunogens of the invention can include substitutions of amino acid residues between the two β strands, β3 and β4. For example, the redesigned immunogen sequences can contain amino acid substitution S182G and/or N183P. Some specific examples of these redesigned RSV soluble F immunogen sequences are shown in SEQ ID NOs: 37, 39, 41 and 43. In some embodiments, the redesigned RSV soluble F immunogens of the invention can have a sequence that is a conservatively modified variant of these exemplified sequences or a substantially identical sequence.

### VI. Nanoparticle Displayed Vaccine Compositions

The invention provides vaccine compositions that contain a heterologous scaffold that display the stabilized soluble F proteins or immunogens of paramyxoviruses described herein. Any heterologous scaffold can be used to present the engineered soluble F proteins or immunogens in the construction of the vaccines of the invention. This includes a virus-like particle (VLP) such as bacteriophage Q_{β} VLP and nanoparticles. Various nanoparticle platforms can be employed in generating the vaccine compositions of the invention. In general, the nanoparticles employed in the invention need to be formed by multiple copies of a single subunit. The nanoparticles are typically ball-like shaped, and/or have rotational symmetry (e.g., with 3-fold and 5-fold axis), e.g., with an icosahedral structure exemplified herein. Additionally or alternatively, the amino-terminus of the particle subunit has to be exposed and in close proximity to the 3-fold axis, and the spacing of three amino-termini has to closely match the spacing of the carboxyl-termini of the displayed trimeric stabilized soluble F protein.

In various embodiments, the employed self-assembling nanoparticles have a diameter of about 25 nm or less (usually assembled from 12, 24, or 60 subunits) and 3-fold axes on the particle surface. Such nanoparticles provide suitable particle platforms to produce multivalent vaccines. In some preferred embodiments, the paramyxovirus immunogen protein or polypeptide can be presented on self-assembling nanoparticles such as self-assembling nanoparticles derived from I3-01 as exemplified herein (I3-01v9b and I3-01v9c). Other examples of nanoparticles suitable for the invention include nanoparticles derived from ferritin (FR) or E2p. Well known and routinely used in the art, ferritin is a globular protein found in all animals, bacteria, and plants. As is well known in the art, it acts primarily to control the rate and location of polynuclear Fe(III)₂O₃ formation through the transportation of hydrated iron ions and protons to and from a mineralized core. The globular form of ferritin is made up of monomeric subunit proteins (also referred to as monomeric ferritin subunits), which are polypeptides having a molecule weight of approximately 17-20 kDa. E2p is a redesigned variant of dihydrolipoyl acyltransferase from Bacillus stearothermophilus that has been shown to self-assemble into thermostable 60-meric nanoparticle. See, e.g., He et al., Nat. Commun. 7:12041, 2016. Similarly, I3-01 is an engineered protein that can self-assemble into hyperstable nanoparticles. See, e.g., Hsia et al., Nature 535, 136-139, 2016. Database search reveals that I3-01 is engineered from a bacterial enzyme with a known crystal structure (PDB ID: 1VLW). Sequences of the subunits of these proteins are known in the art. See, e.g., WO2017/192434. More detailed information on the structural and functional properties of the various nanoparticle scaffolds, as well as their use in presenting trimeric protein immunogens, is provided in the art. See, e.g., WO2017/192434, WO2019/089817 and WO2019/241483. In various embodiments, the paramyxovirus vaccine compositions of the invention can employ any of these known nanoparticles, as well as their conservatively modified variants or variants with substantially identical (e.g., at least 90%, 95% or 99% identical) sequences.

In addition to the nanoparticle sequences noted above, many other nanoparticles or VLPs known in the art may also be used in the practice of the invention. These include, e.g., Aquifex aeolicus lumazine synthase, Thermotoga Maritima encapsulin, Myxococcus xanthus encapsulin, bacteriophage Qbeta virus particle, Flock House Virus (FHV) particle, ORSAY virus particle, and infectious bursal disease virus (IBDV) particle.

Other than the displayed soluble F immunogen, the nanoparticle vaccine compositions of the invention can include additional motifs for better biological or pharmaceutical properties. The additional structural components can function to facilitate the immunogen display on the surface of the nanoparticles, to enhance the stability of the displayed immunogens, and/or to improve yield and purity of the self-assembled protein vaccines. In these embodiments, one or more linkers (linker sequences, motifs or moieties) can be used to connect the various structural components in the constructs. One example of the additional structural components is a trimerization motif such as foldon as noted above. In some embodiments, the coding sequence of a polypeptide fragment or motif that serves as an active site for chemical conjugation can be inserted into the construct at an appropriate position. In some other embodiments, additional structural components such as a CD4⁺ T-helper epitope or a CD8⁺ T-cell epitope can also be inserted into the nanoparticle construct at an appropriate position. These include, e.g., the PADRE T-helper epitope as exemplified herein.

In still some other embodiments, the nanoparticle vaccines of the invention can contain a locking domain that stabilizes the nanoparticle. The locking domain coding sequence can be fused directly or indirectly to the C-terminus of the nanoparticle subunit coding sequence. The locking domain stabilizes the nanoparticles from the inside so that the nanoparticles presenting the paramyxovirus immunogen polypeptide can remain intact during manufacture, vaccine formulation, and immunization. The nanoparticle vaccine immunogens thus constructed have significantly enhanced stability. In general, the locking domain suitable for the invention is a protein subunit that can naturally form a dimer with another protein subunit in solution through non-covalent interactions at the interface. In some preferred embodiments, the two protein subunits can be identical in sequence and form a homodimer. In some other embodiments, the two protein subunits can be different proteins, or two different domains of a single protein derived through engineering, that can form a heterodimer in solution through non-covalent interactions at the interface. Typically, the locking domain is covalently fused to the nanoparticle subunit to which the immunogen polypeptide is linked. Examples of specific locking domains and guidance on the use of a locking domain (e.g., LD7 or LD4 as exemplified herein) in the construction of nanoparticle displayed trimeric immunogens can be found in the art, e.g., WO2019/241483. Two specific locking domains suitable for use in the nanoparticle vaccines of the inventio, LD4 and LD7, are exemplified herein.

Locking domain LD4 (SEQ ID NO: 29):
FSEEQKKALDLAFYFDRRLTPEWRRYLSQRLGLNEEQIERWFRRKEQQI
GWSHPQFEK
Locking domain LD7 (SEQ ID NO: 30):
SPAVDIGDRLDELEKALEALSAEDGHDDVGQRLESLLRRWNSRRAD

Nanoparticles displaying any of the stabilized paramyxovirus soluble F protein immunogens described herein (e.g., stabilized RSV soluble F trimer immunogens) can be constructed by fusing the immunogen polypeptide or subunit of multimeric immunogen protein (e.g., a trimer immunogen) to the subunit sequence of the nanoparticle (e.g., E2p I3-01v9b or I3-01v9c subunit sequence as exemplified herein), as well as the other optional or alternative components described herein (e.g., a locking domain or a trimerization motif). To construct the nanoparticle displayed fusion vaccine immunogens of the invention, one or more linker motifs or moieties may be employed to facilitate connection and maintain structural integrity of the different components. Typically, the linker motifs contain short peptide sequences. In various embodiments, the linkers or linker motifs can be any flexible peptides that connect two protein domains or motifs without interfering with their functions. For example, any of these linkers used in the constructs can be GC-rich peptides with a sequence of (GₐS_{b})ₙ, wherein a is an integer of about 1-5, b is an integer of about 0-2, and n is an integer of about 1-5. In some embodiments, the employed linkers comprise a sequence GSGS (SEQ ID NO:27) or GSGSGSGS (SEQ ID NO:28). Detailed procedures for recombinant production of the vaccine compositions of the invention can be based on the protocols described herein and/or other methods that have been described in the art, e.g., He et al., Nat. Comm. 7, 12041, 2016; Kong et al., Nat. Comm. 7, 12040, 2016; He et al., Sci Adv. 4(11):eaau6769, 2018; WO2017/192434; WO2019/089817 and WO2019/241483.

### VII. Polynucleotides and Expression Constructs

The stabilized paramyxovirus soluble F proteins and the related vaccine compositions of the invention are typically produced by first generating expression constructs (i.e., expression vectors) that contain operably linked coding sequences of the various structural components described herein. Accordingly, in some related aspects, the invention provides substantially purified polynucleotides (DNA or RNA) that encode the nanoparticle displayed immunogens as described herein (e.g., stabilized RSV soluble F immunogens), as well as expression vectors that harbor such polynucleotides (e.g., CMV vectors) and host cells for producing the vaccine immunogens (e.g., HEK293F and ExpiCHO cell lines exemplified herein). The fusion polypeptides encoded by the polynucleotides or expressed from the vectors are also included in the invention. As described herein, such polypeptides will self-assemble into nanoparticle vaccines that display the immunogen polypeptides or proteins on its surface.

The polynucleotides and related vectors can be readily generated with standard molecular biology techniques or the protocols exemplified herein. For example, general protocols for cloning, transfecting, transient gene expression and obtaining stable transfected cell lines are described in the art, e.g., Sambrook et al., Molecular Cloning: A Laboratory Manual, Cold Spring Harbor Press, N.Y., (3^{rd} ed., 2000); and Brent et al., Current Protocols in Molecular Biology, John Wiley & Sons, Inc. (ringbou ed., 2003). Introducing mutations to a polynucleotide sequence by PCR can be performed as described in, e.g., PCR Technology: Principles and Applications for DNA Amplification, H.A. Erlich (Ed.), Freeman Press, NY, NY, 1992; PCR Protocols: A Guide to Methods and Applications, Innis et al. (Ed.), Academic Press, San Diego, CA, 1990; Mattila et al., Nucleic Acids Res. 19:967, 1991; and Eckert et al., PCR Methods and Applications 1:17, 1991.

The selection of a particular vector depends upon the intended use of the fusion polypeptides. For example, the selected vector must be capable of driving expression of the fusion polypeptide in the desired cell type, whether that cell type be prokaryotic or eukaryotic. Many vectors contain sequences allowing both prokaryotic vector replication and eukaryotic expression of operably linked gene sequences. Vectors useful for the invention may be autonomously replicating, that is, the vector exists extrachromosomally and its replication is not necessarily directly linked to the replication of the host cell's genome. Alternatively, the replication of the vector may be linked to the replication of the host's chromosomal DNA, for example, the vector may be integrated into the chromosome of the host cell as achieved by retroviral vectors and in stably transfected cell lines. Both viral-based and nonviral expression vectors can be used to produce the immunogens in a mammalian host cell. Nonviral vectors and systems include plasmids, episomal vectors, typically with an expression cassette for expressing a protein or RNA, and human artificial chromosomes (see, e.g., Harrington et al., Nat. Genet. 15:345, 1997). Useful viral vectors include vectors based on lentiviruses or other retroviruses, adenoviruses, adenoassociated viruses, Cytomegalovirus, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

Depending on the specific vector used for expressing the fusion polypeptide, various known cells or cell lines can be employed in the practice of the invention. The host cell can be any cell into which recombinant vectors carrying a fusion of the invention may be introduced and wherein the vectors are permitted to drive the expression of the fusion polypeptide is useful for the invention. It may be prokaryotic, such as any of a number of bacterial strains, or may be eukaryotic, such as yeast or other fungal cells, insect or amphibian cells, or mammalian cells including, for example, rodent, simian or human cells. Cells expressing the fusion polypeptides of the invention may be primary cultured cells or may be an established cell line. Thus, in addition to the cell lines exemplified herein (e.g., CHO cells), a number of other host cell lines capable well known in the art may also be used in the practice of the invention. These include, e.g., various Cos cell lines, HeLa cells, Sf9 cells, HEK293, AtT20, BV2, and N18 cells, myeloma cell lines, transformed B-cells and hybridomas.

The use of mammalian tissue cell culture to express polypeptides is discussed generally in, e.g., Winnacker, From Genes to Clones, VCH Publishers, N.Y., N.Y., 1987. The fusion polypeptide-expressing vectors may be introduced to the selected host cells by any of a number of suitable methods known to those skilled in the art. For the introduction of fusion polypeptide-encoding vectors to mammalian cells, the method used will depend upon the form of the vector. For plasmid vectors, DNA encoding the fusion polypeptide sequences may be introduced by any of a number of transfection methods, including, for example, lipid-mediated transfection (“lipofection”), DEAE-dextran-mediated transfection, electroporation or calcium phosphate precipitation. These methods are detailed, for example, in Brent et al., supra. Lipofection reagents and methods suitable for transient transfection of a wide variety of transformed and non-transformed or primary cells are widely available, making lipofection an attractive method of introducing constructs to eukaryotic, and particularly mammalian cells in culture. For example, LipofectAMINE™ (Life Technologies) or LipoTaxi™ (Stratagene) kits are available. Other companies offering reagents and methods for lipofection include Bio-Rad Laboratories, CLONTECH, Glen Research, Life Technologies, JBL Scientific, MBI Fermentas, PanVera, Promega, Quantum Biotechnologies, Sigma-Aldrich, and Wako Chemicals USA.

For long-term, high-yield production of recombinant fusion polypeptides, stable expression is preferred. Rather than using expression vectors which contain viral origins of replication, host cells can be transformed with the fusion polypeptide-encoding sequences controlled by appropriate expression control elements (e.g., promoter, enhancer, sequences, transcription terminators, polyadenylation sites, etc.), and selectable markers. The selectable marker in the recombinant vector confers resistance to the selection and allows cells to stably integrate the vector into their chromosomes. Commonly used selectable markers include neo, which confers resistance to the aminoglycoside G-418 (Colberre-Garapin, et al., J. Mol. Biol., 150:1, 1981); and hygro, which confers resistance to hygromycin (Santerre et al., Gene, 30:147, 1984). Through appropriate selections, the transfected cells can contain integrated copies of the fusion polypeptide encoding sequence.

### VIII. Pharmaceutical Compositions and Therapeutic Applications

In another aspect, the invention provides pharmaceutical compositions and related therapeutic methods of using the redesigned paramyxovirus F immunogens and nanoparticle vaccine compositions as described herein. In some embodiments, the soluble F trimer immunogen for the different viruses (e.g., hRSV) can be used for preventing and treating the corresponding viral infections. Some embodiments of the invention relate to use of the hRSV soluble F based vaccines for preventing or treating RSV infections in human subjects. Some embodiments of the invention relate to use of the hMPV soluble F based vaccines for preventing or treating MPV viral infections. Some embodiments of the invention relate to use of the hPIV soluble F based vaccines for preventing or treating PIV viral infections.

In the practice of the various therapeutic methods of the invention, the subjects in need of prevention or treatment of a disease or condition (e.g., hRSV infection) is administered with the corresponding nanoparticle vaccine, the immunogen protein or polypeptide, or an encoding polynucleotide described herein. Typically, the nanoparticle vaccine, the immunogen protein or the encoding polynucleotide disclosed herein is included in a pharmaceutical composition. The pharmaceutical composition can be either a therapeutic formulation or a prophylactic formulation. Typically, the composition can additionally include one or more pharmaceutically acceptable vehicles and, optionally, other therapeutic ingredients (for example, antiviral drugs). Various pharmaceutically acceptable additives can also be used in the compositions.

Thus, some of the pharmaceutical compositions of the invention are vaccine compositions. For vaccine compositions, appropriate adjuvants can be additionally included. Examples of suitable adjuvants include, e.g., aluminum hydroxide, lecithin, Freund's adjuvant, MPL™ and IL-12. In some embodiments, the vaccine compositions or nanoparticle immunogens disclosed herein (e.g., hRSV vaccine composition) can be formulated as a controlled-release or time-release formulation. This can be achieved in a composition that contains a slow release polymer or via a microencapsulated delivery system or bioadhesive gel. The various pharmaceutical compositions can be prepared in accordance with standard procedures well known in the art. See, e.g., Remington's Pharmaceutical Sciences, 19^{th} Ed., Mack Publishing Company, Easton, Pa., 1995; Sustained and Controlled Release Drug Delivery Systems, J. R. Robinson, ed., Marcel Dekker, Inc., New York, 1978); U.S. Pat. Nos. 4,652,441 and 4,917,893; 4,677,191 and 4,728,721; and 4,675,189.

The pharmaceutical compositions of the invention can be readily employed in a variety of therapeutic or prophylactic applications, e.g., for treating hRSV infection or bronchiolitis, or eliciting an immune response to hRSV in a subject. In various embodiments, the vaccine compositions can be used for treating or preventing infections caused by a pathogen from which the displayed immunogen polypeptide in the nanoparticle vaccine is derived. Thus, the vaccine compositions of the invention can be used in diverse clinical settings for treating or preventing infections caused by various viruses. As exemplification, an RSV nanoparticle vaccine composition can be administered to a subject to induce an immune response to hRSV, e.g., to induce production of broadly neutralizing antibodies to the virus. For subjects at risk of developing an RSV infection, a vaccine composition of the invention can be administered to provide prophylactic protection against viral infection. Therapeutic and prophylactic applications of vaccines derived from the other immunogens described herein can be similarly performed. Depending on the specific subject and conditions, pharmaceutical compositions of the invention can be administered to subjects by a variety of administration modes known to the person of ordinary skill in the art, for example, intramuscular, subcutaneous, intravenous, intra-arterial, intra-articular, intraperitoneal, or parenteral routes. In general, the pharmaceutical composition is administered to a subject in need of such treatment for a time and under conditions sufficient to prevent, inhibit, and/or ameliorate a selected disease or condition or one or more symptom(s) thereof. For therapeutic applications, the compositions should contain a therapeutically effective amount of the nanoparticle immunogen described herein. For prophylactic applications, the compositions should contain a prophylactically effective amount of the nanoparticle immunogen described herein. The appropriate amount of the immunogen can be determined based on the specific disease or condition to be treated or prevented, severity, age of the subject, and other personal attributes of the specific subject (e.g., the general state of the subject's health and the robustness of the subject's immune system). Determination of effective dosages is additionally guided with animal model studies followed up by human clinical trials and is guided by administration protocols that significantly reduce the occurrence or severity of targeted disease symptoms or conditions in the subject.

For prophylactic applications, the immunogenic composition is provided in advance of any symptom, for example in advance of infection. The prophylactic administration of the immunogenic compositions serves to prevent or ameliorate any subsequent infection. Thus, in some embodiments, a subject to be treated is one who has, or is at risk for developing, an infection (e.g., RSV infection), for example because of exposure or the possibility of exposure to the virus (e.g., RSV). Following administration of a therapeutically effective amount of the disclosed therapeutic compositions, the subject can be monitored for an infection (e.g., RSV infection), symptoms associated with an infection (e.g., RSV infection), or both.

For therapeutic applications, the immunogenic composition is provided at or after the onset of a symptom of disease or infection, for example after development of a symptom of infection (e.g., RSV infection), or after diagnosis of the infection. The immunogenic composition can thus be provided prior to the anticipated exposure to the virus so as to attenuate the anticipated severity, duration or extent of an infection and/or associated disease symptoms, after exposure or suspected exposure to the virus, or after the actual initiation of an infection. The pharmaceutical composition of the invention can be combined with other agents known in the art for treating or preventing infections by a relevant pathogen (e.g., hRSV infection).

The nanoparticle vaccine compositions containing novel structural components as described in the invention (e.g., hRSV vaccine) or pharmaceutical compositions of the invention can be provided as components of a kit. Optionally, such a kit includes additional components including packaging, instructions and various other reagents, such as buffers, substrates, antibodies or ligands, such as control antibodies or ligands, and detection reagents. An optional instruction sheet can be additionally provided in the kits.

### EXAMPLES

The following examples are offered to illustrate, but not to limit the present invention.

### Example 1 Comparative Analysis of Existing RSV Prefusion F Designs

In this study, we compared three known RSV prefusion F designs, DS-Cav1 (McLellan et al, Science 2013, 342:592-598), SC-TM (Krarup et al., Nat Comm 2015, 6:8143), and sc9-10 DS-Cav1 (Joyce et al., Nat Struct Mol Biol 2016, 23:811-820). Constructs were generated for these three prefusion F designs with an enzymatic site (amino acids “AS”) and a foldon motif attached to the C-terminus. The sequences are shown below.

(SEQ ID NO: 2)
MELLILKANAITTILTAVTFCFASG: N-terminal leader.
(SEQ ID NO: 3)
QSTPPTNNRARR: Unstructured F2 C-terminus.
(SEQ ID NO: 4)
QSTPATNNQAR: F2 C-terminus with mutations.
(SEQ ID NO: 5)
ELPRFMNYTLNNAKKTNVTLSKKRKRR: P27 peptide.
(SEQ ID NO: 6)
FLGFLLGVGS: fusion peptide (FP).
(SEQ ID NO: 7)
GYIPEAPRDGQAYVRKDGEWVLLSTFL: C-terminal foldon.
A_DS-Cav1-foldon (PDB ID: 4MMU)
(SEQ ID NO: 8)
MELLILKANAITTILTAVTFCFASGQNITEEFYQSTCSAVSKGYLSALRTGWY
TSVITIELSNIKENKCNGTDAKVKLIKQELDKYKNAVTELQLLMQSTPATNNRA
RRFLGFLLGVGSAIASGVAVCKVLHLEGEVNKIKSALLSTNKAVVSLSNGVSVL
TFKVLDLKNYIDKQLLPILNKQSCSISNIETVIEFQQKNNRLLEITREFSVNAGVT
TPVSTYMLTNSELLSLINDMPITNDQKKLMSNNVQIVRQQSYSIMCIIKEEVLAY
VVQLPLYGVIDTPCWKLHTSPLCTTNTKEGSNICLTRTDRGWYCDNAGSVSFFP
QAETCKVQSNRVFCDTMNSLTLPSEVNLCNVDIFNPKYDCKIMTSKTDVSSSVI
TSLGAIVSCYGKTKCTASNKNRGIIKTFSNGCDYVSNKGVDTVSVGNTLYYVN
KQEGKSLYVKGEPIINFYDPLVFPSDEFDASISQVNEKINQSLAFIRKSDELLASG
YIPEAPRDGQAYVRKDGEWVLLSTFL
A2_SC-TM-foldon (PDB ID: 5C6B)
(SEQ ID NO: 9)
MELLILKANAITTILTAVTFCFASGQNITEEFYQSTCSAVSKGYLSALRTGWY
TSVITIELSNIKKIKCNGTDAKIKLIKQELDKYKNAVTELQLLMQSTPATNNQA
RGSGSGRSLGFLLGVGSAIASGVAVSKVLHLEGEVNKIKSALLSTNKAVVSLSN
GVSVLTSKVLDLKNYIDKQLLPIVNKQSCSIPNIETVIEFQQKNNRLLEITREFSV
NAGVTTPVSTYMLTNSELLSLINDMPITNDQKKLMSNNVQIVRQQSYSIMSIIKE
EVLAYVVQLPLYGVIDTPCWKLHTSPLCTTNTKEGSNICLTRTDRGWYCDNAG
SVSFFPQAETCKVQSNRVFCDTMNSLTLPSEVNLCNVDIFNPKYDCKIMTSKTD
VSSSVITSLGAIVSCYGKTKCTASNKNRGIIKTFSNGCDYVSNKGVDTVSVGNT
LYYVNKQEGKSLYVKGEPIINFYDPLVFPSDQFDASISQVNEKINQSLAFIRKSD
ELLASGYIPEAPRDGQAYVRKDGEWVLLSTFL
A2_sc9-10 DS-Cav1-foldon (PDB ID: 5K61)
(SEQ ID NO: 10)
MELLILKANAITTILTAVTFCFASGQNITEEFYQSTCSAVSKGYLGALRTGWY
TSVITIELSNIKENKCNGTDAKVKLIKQELDKYKNAVTDLQLLMQSTPATGSGS
AICSGVAVCKVLHLEGEVNKIKSALLSTNKAVVSLSNGVSVLTFKVLDLKNYI
DKQLLPILNKQSCSIPNIETVIEFQQKNNRLLEITREFSVNAGVTTPVSTYMLINS
ELLSLINDMPITNDQKKLMSNNVQIVRQQSYSIMCIIKEEVLAYVVQLPLYGVID
TPCWKLHTSPLCTTNTKEGSNICLTRTDRGWYCDNAGSVSFFPQAETCKVQSN
RVFCDTMNSRTLPSEVNLCNVDIFNPKYDCKIMTSKTDVSSSVITSLGAIVSCYG
KTKCTASNKNRGIIKTFSNGCDYVSNKGVDTVSVGNTLYCVNKQEGQSLYVK
GEPIINFYDPLVFPSDEFDASISQVNEKINQSLAFIRKSDELLASGYIPEAPRDGQA
YVRKDGEWVLLSTFL

These three constructs were transiently expressed in 25 ml ExpiCHO cells and purified using a D25 antibody column followed by size-exclusion chromatography (SEC) on a Superdex 200 Increase 100/300 GL column. The use of D25, a potent neutralizing antibody (NAb) that targets site-Ø (McLellan et al, Science 2013, 342:592-598), will ensure prefusion-specific RSV F purification. For DS-Cav1, the 3.0 Å-resolution crystal structure showed a football-shaped, closed prefusion F trimer conformation. Transient expression of DS-Cav1 in ExpiCHO cells produced reasonable yield after D25 purification. However, the SEC profile showed a high aggregation peak (at ~9 ml) and a second peak mainly corresponding to trimers. Of note, we observed a visible shoulder left to the trimer peak in SEC, suggesting the presence of higher-order prefusion F species in the DS-Cav1 sample. Negative-stain EM (nsEM) was performed to characterize the trimer fractions (around 12 ml). All 2D classes showed monomers or dimers without any sign of closed prefusion trimers. For SC-TM, the 2.4 Å-resolution crystal structure showed a similar closed prefusion trimer to DS-Cav1. Notably, SC-TM had an extremely low yield in ExpiCHO expression and exhibited both a trimer peak and a monomer leak in SEC. The 2D class images from nsEM showed football-shaped molecules characteristic of closed prefusion F trimers, as well as monomers and dimers. We used these 2D classes to construct a 3D EM model, which matches the crystal structure nearly perfectly and shows unoccupied density at the trimer bottom corresponding to the C-terminal foldon. However, we also found wedge-shaped molecules in the nsEM images corresponding to the postfusion F trimers, suggesting that SC-TM cannot prevent the pre-to-post conformational transition.

For sc9-10 DS-Cav1, the 2.9 Å-resolution crystal structure showed a closed prefusion F trimer almost identical to DS-Cav1 and SC-TM. The sc9-10 DS-Cav1 construct resulted in high yield and high purity in SEC, showing a >10 and a >250 times higher trimer peaks than DS-Cav1 and SC-TM, respectively. In the nsEM analysis, almost all 2D classes showed “football” shapes, suggesting close to 100% closed prefusion F trimers. A 3D structure model constructed from the EM data confirmed this finding. In summary, DS-Cav1 becomes monomeric in solution, SC-TM expresses prefusion monomers and closed trimers, as well as postfusion trimers with little yield, and sc9-10 DS-Cav1 produces closed prefusion trimers with high yield and high purity.

### Example 2 Analysis of Sources of RSV F Metastability

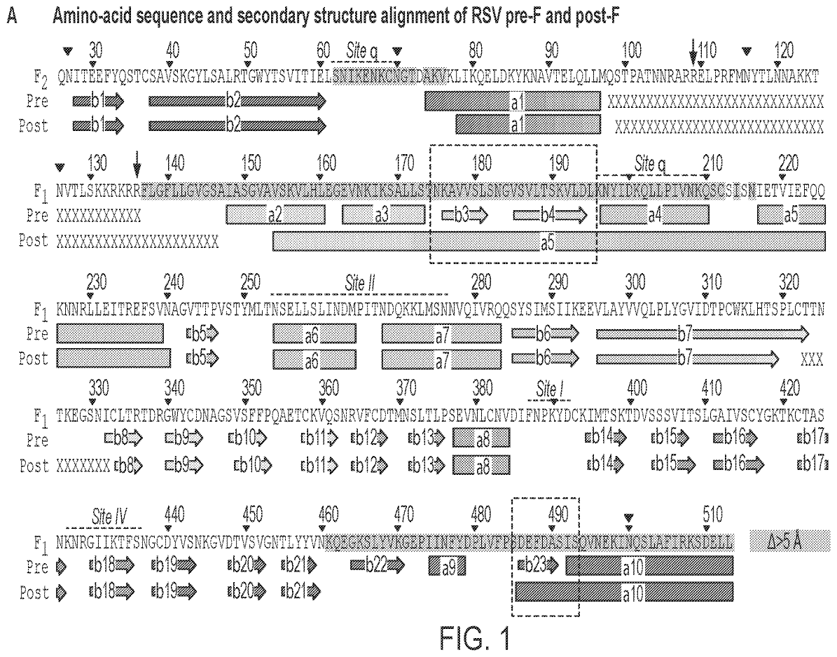

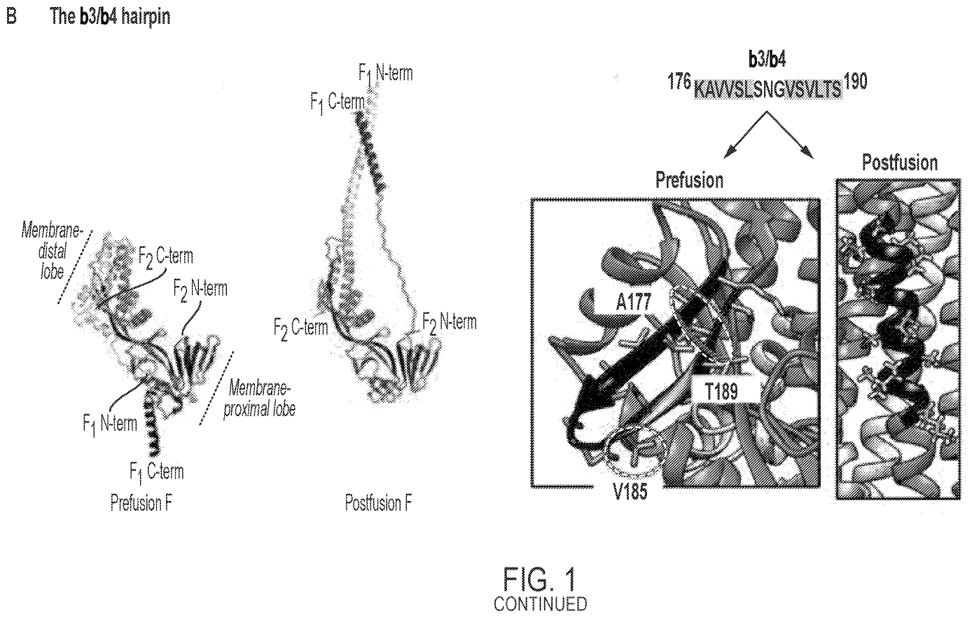

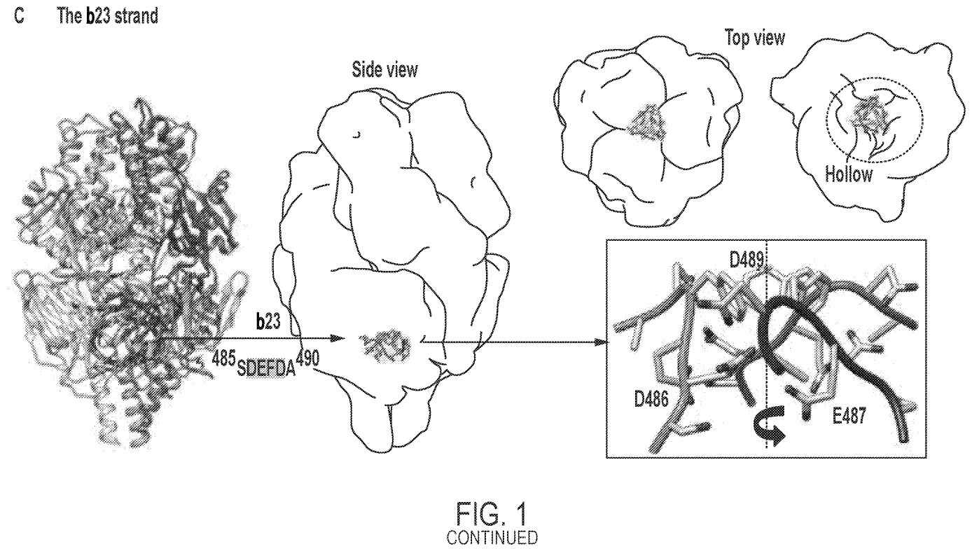

We analyzed the sequences and structures of RSV F to identify the potential causes of F metastability. The RSV strain A2 (GenBank ID: AAB59858.1, UniProt ID: P03420), which was previously used to design prefusion F constructs, was used as a template in this study. Briefly, the sequence and secondary structure alignment of A2 F in the prefusion and postfusion states revealed several regions of importance (FIG. 1, A). Two such regions are β3/β4 and β23, which both undergo secondary structure changes and become α-helices in the postfusion conformation.

For the β3/β4 segment (K176-S190), it appears as a β-hairpin in the prefusion state but becomes part of an extended «-helix in the postfusion state (FIG. 1, B). Structural analysis suggested that disulfide bonds S180C/S186C and A177C/T189C, with Cβ-Cβ distances of 4.25 Å and 4.91 Å, respectively, may stabilize RSV F in a prefusion state (this has been covered in our previous paramyxovirus patent filing). Here, we report another mutation, V185P. In the unmutated prefusion F (PDB ID: 4JHW), the backbone dihedral angles of V185 are −70.6 (Phi) and 126.4 (Psi), which closely match those of a trans-proline: −75 (Phi) and 145 (Psi). We hypothesize that V185P can rigidify the prefusion hairpin structure but introduce a kink in the postfusion helix, thus destabilizing the postfusion conformation (FIG. 1, B, right). For the β23 segment (S485-A490), it forms interactions with β23 segments of other two protomers around the 3-fold axis and sits above the trimeric coiled coil formed by three α10 helices, which hold the three F protomers in a trimeric conformation (FIG. 1, C, left). A cross-section analysis revealed that the β23 cluster is located at the bottom the hollow interior of the football-shaped RSV prefusion F trimer (FIG. 1, C, right top). For the wildtype RSV prefusion F trimer, this hollow interior is partially filled by the fusion peptides of three F protomers but will become empty once the fusion peptides are removed, e.g., in the prefusion F design sc9-10 DS-Cav1. Further analysis of the β23 cluster revealed an unusual pattern of interactions (FIG. 1, C, right bottom). To be more specific, the short β23 strand contains three negatively charged residues, D486, E487, and D489, which form repulsive charge-charge interactions around the 3-fold trimer axis. We hypothesize that the unfavorable interactions in the β23 cluster can facilitate rapid opening of the wildtype prefusion F trimer on the RSV virion surface to expose the fusion peptides and to accelerate the pre-to-post conformational change during cell entry. In another word, we hypothesize that the β23 cluster is a major cause of RSV F metastability. In this study, we tested our hypothesis by mutating D486 and E487 to (1) polar residues, e.g., D486N and E487Q, which can form salt bridges, and (2) hydrophobic residues, e.g., D486L and E487L, which can form hydrophobic clusters. Other mutations to the β23 segment may further improve trimer stability.

### Example 3 Characterization of the RSV Prefusion F Constructs Based on “V2-Ext-PDB6-D”

We generated five soluble F constructs using “V2-Ext-PDB6-D” as the base design, all containing a C-terminal foldon motif (sequences listed below). The 1^{st} construct is the base design. The 2^{nd} construct incorporates the V185P mutation into the base design to examine the effect of the second proline mutation, V185P. The 3^{rd} and 4^{th} constructs incorporate mutations D486N/E487Q and D486L/E487L into the 2^{nd} construct, respectively, to examine whether removal of the repulsive charge-charge interactions at β23 can improve the stability of RSV prefusion F trimer. The 5^{th} construct incorporates the A149C/Y458C mutation (termed SS4) into the base design to test this inter-protomer disulfide bond in combination with a minimal set of mutations in the base design. This disulfide bond was used in sc9-10 DS-Cav1 (Joyce et al., Nat Struct Mol Biol 2016, 23:811-820). Therefore, the 5^{th} construct, which uses a covalent bond to lock RSV prefusion F in a closed trimer, provides a “positive control” for the 3^{rd} and 4^{th} constructs, which attempt to retain RSV prefusion F in a closed trimer through engineered non-covalent interactions at β23.

A2_V2-Ext-PDB6-D-foldon (S215P, DB6 = S155-S290, E92D)
(SEQ ID NO: 11)
MELLILKANAITTILTAVTFCFASGQNITEEFYQSTCSAVSKGYLSALRTGWY
TSVITIELSNIKENKCNGTDAKVKLIKQELDKYKNAVTDLQLLMQSTPATGSGS
AIASGVAVCKVLHLEGEVNKIKSALLSTNKAVVSLSNGVSVLTSKVLDLKNYI
DKQLLPIVNKQSCSIPNIETVIEFQQKNNRLLEITREFSVNAGVTTPVSTYMLTNS
ELLSLINDMPITNDQKKLMSNNVQIVRQQSYSIMCIIKEEVLAYVVQLPLYGVID
TPCWKLHTSPLCTTNTKEGSNICLTRTDRGWYCDNAGSVSFFPQAETCKVQSN
RVFCDTMNSLTLPSEVNLCNVDIFNPKYDCKIMTSKTDVSSSVITSLGAIVSCYG
KTKCTASNKNRGIIKTFSNGCDYVSNKGVDTVSVGNTLYYVNKQEGKSLYVK
GEPIINFYDPLVFPSDEFDASISQVNEKINQSLAFIRKSDELLASGYIPEAPRDGQA
YVRKDGEWVLLSTFL
GA2 V2-Ext-P2DB6-D-foldon (S215P, DB6 = S155-S290, E92D, V185P)
(SEQ ID NO: 12)
MELLILKANAITTILTAVTFCFASGQNITEEFYQSTCSAVSKGYLSALRTGWY
TSVITIELSNIKENKCNGTDAKVKLIKQELDKYKNAVTDLQLLMQSTPATGSGS
AIASGVAVCKVLHLEGEVNKIKSALLSTNKAVVSLSNGPSVLTSKVLDLKNYID
KQLLPIVNKQSCSIPNIETVIEFQQKNNRLLEITREFSVNAGVTTPVSTYMLTNSE
LLSLINDMPITNDQKKLMSNNVQIVRQQSYSIMCIIKEEVLAYVVQLPLYGVIDT
PCWKLHTSPLCTTNTKEGSNICLTRTDRGWYCDNAGSVSFFPQAETCKVQSNR
VFCDTMNSLTLPSEVNLCNVDIFNPKYDCKIMTSKTDVSSSVITSLGAIVSCYGK
TKCTASNKNRGIIKTFSNGCDYVSNKGVDTVSVGNTLYYVNKQEGKSLYVKG
EPIINFYDPLVFPSDEFDASISQVNEKINQSLAFIRKSDELLASGYIPEAPRDGQAY
VRKDGEWVLLSTFL
A2_V2-Ext-P2DB6-D-NQ without N-terminal leader and C-terminal
foldon (S215P, DB6 = S155-S290, E92D, V185P, D486N + E487Q)
(SEQ ID NO: 17)
QNITEEFYQSTCSAVSKGYLSALRTGWYTSVITIELSNIKENKCNGTDAKVKLIK
QELDKYKNAVTDLQLLMQSTPATGSGSAIASGVAVCKVLHLEGEVNKIKSALL
STNKAVVSLSNGPSVLTSKVLDLKNYIDKQLLPIVNKQSCSIPNIETVIEFQQKN
NRLLEITREFSVNAGVTTPVSTYMLTNSELLSLINDMPITNDQKKLMSNNVQIV
RQQSYSIMCIIKEEVLAYVVQLPLYGVIDTPCWKLHTSPLCTTNTKEGSNICLTR
TDRGWYCDNAGSVSFFPQAETCKVQSNRVFCDTMNSLTLPSEVNLCNVDIFNP
KYDCKIMTSKTDVSSSVITSLGAIVSCYGKTKCTASNKNRGIIKTFSNGCDYVSN
KGVDTVSVGNTLYYVNKQEGKSLYVKGEPIINFYDPLVFPSNQFDASISQVNEK
INQSLAFIRKSDELL
A2 V2-Ext-P2DB6-D-L2 without N-terminal leader and C-terminal
foldon (S215P, DB6 = S155-S290, E92D, V185P, D486L + E487L)
(SEQ ID NO: 18)
QNITEEFYQSTCSAVSKGYLSALRTGWYTSVITIELSNIKENKCNGTDAKVKLIK
QELDKYKNAVTDLQLLMQSTPATGSGSAIASGVAVCKVLHLEGEVNKIKSALL
STNKAVVSLSNGPSVLTSKVLDLKNYIDKQLLPIVNKQSCSIPNIETVIEFQQKN
NRLLEITREFSVNAGVTTPVSTYMLTNSELLSLINDMPITNDQKKLMSNNVQIV
RQQSYSIMCIIKEEVLAYVVQLPLYGVIDTPCWKLHTSPLCTTNTKEGSNICLTR
TDRGWYCDNAGSVSFFPQAETCKVQSNRVFCDTMNSLTLPSEVNLCNVDIFNP
KYDCKIMTSKTDVSSSVITSLGAIVSCYGKTKCTASNKNRGIIKTFSNGCDYVSN
KGVDTVSVGNTLYYVNKQEGKSLYVKGEPIINFYDPLVFPSLLFDASISQVNEK
INQSLAFIRKSDELL
A2_V2-Ext-PDB6-D-SS4-foldon (S215P, DB6 = S155-S290, E92D,
SS4 = A149-Y458)
(SEQ ID NO: 13)
MELLILKANAITTILTAVTFCFASGQNITEEFYQSTCSAVSKGYLSALRTGWY
TSVITIELSNIKENKCNGTDAKVKLIKQELDKYKNAVTDLQLLMQSTPATGSGS
AICSGVAVCKVLHLEGEVNKIKSALLSTNKAVVSLSNGPSVLTSKVLDLKNYID
KQLLPIVNKQSCSIPNIETVIEFQQKNNRLLEITREFSVNAGVTTPVSTYMLTNSE
LLSLINDMPITNDQKKLMSNNVQIVRQQSYSIMCIIKEEVLAYVVQLPLYGVIDT
PCWKLHTSPLCTTNTKEGSNICLTRTDRGWYCDNAGSVSFFPQAETCKVQSNR
VFCDTMNSLTLPSEVNLCNVDIFNPKYDCKIMTSKTDVSSSVITSLGAIVSCYGK
TKCTASNKNRGIIKTFSNGCDYVSNKGVDTVSVGNTLYCVNKQEGKSLYVKG
EPIINFYDPLVFPSDEFDASISQVNEKINQSLAFIRKSDELLASGYIPEAPRDGQAY
VRKDGEWVLLSTFL

The five soluble F constructs in the “V2-Ext-PDB6-D” series were transiently expressed in 25 ml ExpiCHO cells and purified using a D25 column followed by SEC on a Superdex 200 Increase 100/300 GL column. For the 1st construct or the base design, A2_V2-Ext-PDB6-D-foldon, we observed a single trimer peak in SEC with high yield and high purity. However, the nsEM analysis of the SEC fraction around ~12 ml showed predominantly “open” prefusion F trimers, with two classes showing closed prefusion trimers. The 3D EM model constructed from these two classes closely matches the prefusion F crystal structure. Our results thus demonstrate that this base design—with a minimum set of mutations—can produce prefusion F with a small fraction of closed trimers. For the 2^{nd} construct, A2_V2-Ext-P2DB6-D-foldon, we observed similar profiles to the base design, suggesting that the second proline mutation, V185P, may have little effect on protein properties. For the 3^{rd} construct, A2_V2-Ext-P2DB6-D-NQ-foldon, we observed distinct features compared to the 2^{nd} construct, which does not contain the D486N/E487Q mutation. To be more specific, A2_V2-Ext-P2DB6-D-NQ-foldon produces a single trimer peak with high yield and high purity, but with a slightly increased aggregate peak around ~8.5 ml. Remarkably, the nsEM analysis revealed that many 2D classes, or ~73% of molecules, correspond to closed prefusion F trimers. The 3D EM models constructed from the EM data match the prefusion F crystal structure with nearly perfect fitting, with some variations around the α10 helices at the trimer bottom. For the 4^{th} construct, A2_V2-Ext-P2DB6-D-L2-foldon, we observed overall similar properties to the 3^{rd} construct with a slightly higher ratio of closed prefusion F trimers, 76% vs. 73%. For the 5^{th} construct, A2_V2-Ext-PDB6-D-SS4-foldon, we observed a trimer peak with high yield and high purity, but with a further increase in aggregation compared with the 3^{rd} and 4^{th} constructs. The nsEM analysis revealed the highest percentage of closed prefusion F trimers, 88%, among all the constructs in this series. The 3D EM model not only matches the crystal structure of the prefusion F trimer but also shows density for the C-terminal foldon domain. In summary, our results from constructs #3 and #4 support the hypothesis that the β23 strand is a major cause of RSV F metastability and removal of the repulsive change-charge interactions at β23 significantly improves trimer stability at a similar level to a well-placed inter-protomer disulfide bond. All five constructs, especially constructs #3, #4, and #5 with 73-88% closed trimers, can be developed towards RSV F trimer vaccines. The presence of open trimers should not be a major concern because wildtype prefusion F must be in an equilibrium stage of “closed” and “open” trimers on the RSV virion surface and both states can elicit neutralizing antibodies to block virus entry.

The 3^{rd} and 4^{th} constructs, both containing the engineered non-covalent mutations at β23, were further probed using known RSV antibodies. First, these two constructs were probed using ADI14359, a postfusion-specific antibody. The nsEM analysis revealed that three types of 2D class images correspond to unbound ADI14359 Fabs, open prefusion F trimers or F monomers without bound Fabs, and closed prefusion F trimers without bound Fabs. We did not find any 2D classes corresponding to postfusion F and postfusion F/ADI14359 complex in this analysis. A perfect 3D model of closed prefusion F trimer was constructed, suggesting that ADI14359 does not perturb these two prefusion F constructs and does not trigger any conformational changes in them. Then, these two constructs were probed using D25, a prefusion-specific antibody, which is also the antibody used to purify prefusion F protein in this study. The nsEM analysis revealed that three types of 2D class images correspond to unbound D25 Fabs, open prefusion F trimers or F monomers bound to D25, and closed prefusion F trimers bound to D25. A nearly perfect 3D structure model of closed prefusion F trimer in complex with D25 was constructed from the EM data, providing strong evidence that A2_V2-Ext-P2DB6-D-NQ-foldon and A2_V2-Ext-P2DB6-D-L2-foldon are ideal candidates for RSV prefusion F trimer-based vaccine development.

### Example 4 Characterization of the RSV Prefusion F Constructs Based on the “V2-Ext-PDB6-GDQ” Base

We generated eight soluble F constructs using “V2-Ext-PDB6-GDQ” as the base design, all containing a C-terminal foldon motif (sequences listed below). The 1st construct is the base design that combines V2-Ext-PDB6-D with S46G and K465Q. We hypothesize that the S46G/K465Q mutation can reduce aggregation for some “V2-Ext-PDB6-D” derivatives. The 2^{nd} construct incorporates a second proline mutation, V185P, into the base design. The 3^{rd} and 4^{th} constructs incorporate the D486N/E487Q mutation into the base design, but with the 4^{th} construct containing the V185P mutation. The 5^{th} and 6^{th} constructs incorporate the D486L/E487L mutation into the base design, but with the 6th construct containing the V185P mutation. The 7^{th} construct incorporates the A149C/Y458C mutation (termed SS4) to the base design. This inter-protomer disulfide bond was used in sc9-10 DS-Cav1 (Joyce et al., Nat Struct Mol Biol 2016, 23:811-820). Therefore, the 7^{th} construct, which uses a covalent bond to lock RSV prefusion F in a closed trimer, provides a “positive control” for the 3^{rd}-6^{th} constructs, which attempt to retain RSV prefusion F in a closed trimer through engineered non-covalent interactions at β23. The 8^{th} construct is designed to examine whether the inter-protomer disulfide bond, namely A149C/Y458C, and the polar mutations at β23, namely D486N/E487Q, can be combined into one construct to further stabilize the prefusion F trimer.

A2_V2-Ext-PDB6-GDQ-foldon (S215P, DB6 = S155-S290,
S46G + E92D + K465Q)
(SEQ ID NO: 14)
MELLILKANAITTILTAVTFCFASG-
QNITEEFYQSTCSAVSKGYLGALRTGWYTSVITIELSNIKENKCNGTDAKVKLI
KQELDKYKNAVTDLQLLMQSTPATGSGSAIASGVAVCKVLHLEGEVNKIKSAL
LSTNKAVVSLSNGVSVLTSKVLDLKNYIDKQLLPIVNKQSCSIPNIETVIEFQQK
NNRLLEITREFSVNAGVTTPVSTYMLTNSELLSLINDMPITNDQKKLMSNNVQI
VRQQSYSIMCIIKEEVLAYVVQLPLYGVIDTPCWKLHTSPLCTTNTKEGSNICLT
RTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVS
NKGVDTVSVGNTLYYVNKQEGQSLYVKGEPIINFYDPLVFPSDEFDASISQVNE
KINQSLAFIRKSDELL-ASGYIPEAPRDGQAYVRKDGEWVLLSTFL
A2_V2-Ext-PDB6-GDQ-foldon (S215P, DB6 = S155-S290,
S46G + E92D + K465Q, V185P)
(SEQ ID NO: 15)
MELLILKANAITTILTAVTFCFASG-
QNITEEFYQSTCSAVSKGYLGALRTGWYTSVITIELSNIKENKCNGTDAKVKLI
KQELDKYKNAVTDLQLLMQSTPATGSGSAIASGVAVCKVLHLEGEVNKIKSAL
LSTNKAVVSLSNGPSVLTSKVLDLKNYIDKQLLPIVNKQSCSIPNIETVIEFQQK
NNRLLEITREFSVNAGVTTPVSTYMLTNSELLSLINDMPITNDQKKLMSNNVQI
VRQQSYSIMCIIKEEVLAYVVQLPLYGVIDTPCWKLHTSPLCTTNTKEGSNICLT
RTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVS
NKGVDTVSVGNTLYYVNKQEGQSLYVKGEPIINFYDPLVFPSDEFDASISQVNE
KINQSLAFIRKSDELL-ASGYIPEAPRDGQAYVRKDGEWVLLSTFL
A2_V2-Ext-PDB6-GDQ-NQ without N-terminal leader and C-terminal
foldon (S215P, DB6 = S155-S290, S46G + E92D + K465Q, D486N +
E487Q)
(SEQ ID NO: 19)
QNITEEFYQSTCSAVSKGYLGALRTGWYTSVITIELSNIKENKCNGTDAKVKLI
KQELDKYKNAVTDLQLLMQSTPATGSGSAIASGVAVCKVLHLEGEVNKIKSAL
LSTNKAVVSLSNGVSVLTSKVLDLKNYIDKQLLPIVNKQSCSIPNIETVIEFQQK
NNRLLEITREFSVNAGVTTPVSTYMLTNSELLSLINDMPITNDQKKLMSNNVQI
VRQQSYSIMCIIKEEVLAYVVQLPLYGVIDTPCWKLHTSPLCTTNTKEGSNICLT
RTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVS
NKGVDTVSVGNTLYYVNKQEGQSLYVKGEPIINFYDPLVFPSNQFDASISQVNE
KINQSLAFIRKSDELL
A2_V2-Ext-P2DB6-GDQ-NQ without N-terminal leader and C-terminal
foldon (S215P, DB6 = S155-S290, S46G + E92D + K465Q, V185P,
D486N + E487Q)
(SEQ ID NO: 20)
QNITEEFYQSTCSAVSKGYLGALRTGWYTSVITIELSNIKENKCNGTDAKVKLI
KQELDKYKNAVTDLQLLMQSTPATGSGSAIASGVAVCKVLHLEGEVNKIKSAL
LSTNKAVVSLSNGPSVLTSKVLDLKNYIDKQLLPIVNKQSCSIPNIETVIEFQQK
NNRLLEITREFSVNAGVTTPVSTYMLTNSELLSLINDMPITNDQKKLMSNNVQI
VRQQSYSIMCIIKEEVLAYVVQLPLYGVIDTPCWKLHTSPLCTTNTKEGSNICLT
RTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVS
NKGVDTVSVGNTLYYVNKQEGQSLYVKGEPIINFYDPLVFPSNQFDASISQVNE
KINQSLAFIRKSDELL
A2_V2-Ext-PDB6-GDQ-L2 without N-terminal leader and C-terminal
foldon (S215P, DB6 = S155-S290, S46G + E92D + K465Q, D486L +
E487L)
(SEQ ID NO: 21)
QNITEEFYQSTCSAVSKGYLGALRTGWYTSVITIELSNIKENKCNGTDAKVKLI
KQELDKYKNAVTDLQLLMQSTPATGSGSAIASGVAVCKVLHLEGEVNKIKSAL
LSTNKAVVSLSNGVSVLTSKVLDLKNYIDKQLLPIVNKQSCSIPNIETVIEFQQK
NNRLLEITREFSVNAGVTTPVSTYMLTNSELLSLINDMPITNDQKKLMSNNVQI
VRQQSYSIMCIIKEEVLAYVVQLPLYGVIDTPCWKLHTSPLCTTNTKEGSNICLT
RTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVS
NKGVDTVSVGNTLYYVNKQEGQSLYVKGEPIINFYDPLVFPSLLFDASISQVNE
KINQSLAFIRKSDELL
A2_V2-Ext-P2DB6-GDQ-L2 without N-terminal leader and C-terminal
foldon (S215P, DB6 = S155-S290, S46G + E92D + K465Q, V185P,
D486L + E487L)
(SEQ ID NO: 22)
QNITEEFYQSTCSAVSKGYLGALRTGWYTSVITIELSNIKENKCNGTDAKVKLI
KQELDKYKNAVTDLQLLMQSTPATGSGSAIASGVAVCKVLHLEGEVNKIKSAL
LSTNKAVVSLSNGPSVLTSKVLDLKNYIDKQLLPIVNKQSCSIPNIETVIEFQQK
NNRLLEITREFSVNAGVTTPVSTYMLTNSELLSLINDMPITNDQKKLMSNNVQI
VRQQSYSIMCIIKEEVLAYVVQLPLYGVIDTPCWKLHTSPLCTTNTKEGSNICLT
RTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVS
NKGVDTVSVGNTLYYVNKQEGQSLYVKGEPIINFYDPLVFPSLLFDASISQVNE
KINQSLAFIRKSDELL-
A2_V2-Ext-PDB6-GDQ-SS4-foldon (S215P, DB6 = S155-S290,
S46G + E92D + K465Q, SS4 = A149-Y458)
(SEQ ID NO: 16)
MELLILKANAITTILTAVTFCFASG-
QNITEEFYQSTCSAVSKGYLGALRTGWYTSVITIELSNIKENKCNGTDAKVKLI
KQELDKYKNAVTDLQLLMQSTPATGSGSAICSGVAVCKVLHLEGEVNKIKSAL
LSTNKAVVSLSNGVSVLTSKVLDLKNYIDKQLLPIVNKQSCSIPNIETVIEFQQK
NNRLLEITREFSVNAGVTTPVSTYMLTNSELLSLINDMPITNDQKKLMSNNVQI
VRQQSYSIMCIIKEEVLAYVVQLPLYGVIDTPCWKLHTSPLCTTNTKEGSNICLT
RTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVS
NKGVDTVSVGNTLYCVNKQEGQSLYVKGEPIINFYDPLVFPSDEFDASISQVNE
KINQSLAFIRKSDELL-
ASGYIPEAPRDGQAYVRKDGEWVLLSTFL
A2_V2-Ext-P2DB6-GDQ-SS4-NQ without N-terminal leader and C-
terminal foldon (S215P, DB6 = S155-S290, S46G + E92D + K465Q,
V185P, D486N + E487Q, SS4 = A149-Y458)
(SEQ ID NO: 23)
QNITEEFYQSTCSAVSKGYLGALRTGWYTSVITIELSNIKENKCNGTDAKVKLI
KQELDKYKNAVTDLQLLMQSTPATGSGSAICSGVAVCKVLHLEGEVNKIKSAL
LSTNKAVVSLSNGPSVLTSKVLDLKNYIDKQLLPIVNKQSCSIPNIETVIEFQQK
NNRLLEITREFSVNAGVTTPVSTYMLTNSELLSLINDMPITNDQKKLMSNNVQI
VRQQSYSIMCIIKEEVLAYVVQLPLYGVIDTPCWKLHTSPLCTTNTKEGSNICLT
RTDRGWYCDNAGSVSFFPQAETCKVQSNRVFCDTMNSLTLPSEVNLCNVDIFN
PKYDCKIMTSKTDVSSSVITSLGAIVSCYGKTKCTASNKNRGIIKTFSNGCDYVS
NKGVDTVSVGNTLYCVNKQEGQSLYVKGEPIINFYDPLVFPSNQFDASISQVNE
KINQSLAFIRKSDELL

The seven soluble F constructs in the “V2-Ext-PDB6-GDQ” series were transiently expressed in 25 ml ExpiCHO cells and purified using a D25 column followed by SEC on a Superdex 200 Increase 100/300 GL column. For the 1st construct or the base design, A2_V2-Ext-PDB6-GDQ-foldon, we observed a trimer peak with high yield and high purity but also a monomer peak in SEC. The nsEM analysis of the SEC fraction at ~11.5 ml showed “open” prefusion F with no classes corresponding to “closed” trimers. Our results thus revealed that mutations S46G and K465Q (likely S46G), while keeping RSV F in the prefusion state, tend to shift the equilibrium to the “open” conformation. For the 2^{nd} construct, A2_V2-Ext-P2DB6-GDQ-foldon, we observed similar profiles to the base design. Of note, the slightly abnormal trimer peak was likely caused by the high yield of this construct, and similar pattern has been found elsewhere. For the 3^{rd} and 4^{th} constructs, A2_V2-Ext-PDB6-GDQ-NQ-foldon and A2_V2-Ext-P2DB6-GDQ-NQ-foldon, we observed substantial trimer yield and purity without any aggregate peak in the SEC profiles. The nsEM analysis revealed that most of the trimers are open, with 12% and 6% closed trimers observed for the 3^{rd} and 4^{th} constructs, respectively. The 3D structural models built from the EM data further confirmed that these two constructs could form closed prefusion F trimers.

For the 5^{th} and 6^{th} constructs, A2_V2-Ext-PDB6-GDQ-L2-foldon and A2_V2-Ext-P2DB6-GDQ-L2-foldon, we observed high trimer yield and purity without any aggregate peak in the SEC profiles. The nsEM analysis revealed that majority of the trimers are open, with ~29% closed trimers observed for both constructs. The 3D structural models confirmed that these two constructs could form closed prefusion F trimers. For the 7^{th} construct, incorporation of the inter-protomer disulfide bond into the “V2-Ext-PDB6-GDQ” base resulted in a slight increase of aggregation in the SEC profile. The nsEM analysis showed that 76% of the molecules are closed prefusion F trimers, which was >10% less than when this disulfide bond was incorporated into the V2-Ext-PDB6-D base, 88%. Nonetheless, a nearly perfect 3D structural model was built from the EM data. For the 8^{th} construct, incorporation of both the inter-protomer disulfide bond SS4 and the non-covalent β23 mutation into the “V2-Ext-PDB6-GDQ” base led to further increased aggregation but did not increase the ratio of closed prefusion trimers in the nsEM analysis, with 73% of the molecules corresponding to closed prefusion trimers. A nearly perfect 3D structural model was built from the EM data. In summary, our results from this systematic comparison supported the hypothesis that the S46G/K465 mutation can minimize aggregation in prefusion F expression. However, we noted an adverse effect associated with the S46G/K465 mutation, which is the reduced ratio of “closed” prefusion F trimers. Furthermore, the constructs containing the D486N/E487Q (or “NQ”) mutation appeared to be more sensitive to the S46G/K465 mutation than those containing the D486L/E487L (or “L2”) mutation, suggesting that at β23 salt bridges formed by polar residues are less effective than hydrophobic contacts in holding prefusion F in a closed trimer.

### Example 5 Characterization of Various RSV Prefusion F Constructs by x-Ray Crystallography

Eleven F constructs were characterized structurally by x-ray crystallography. First, crystal structures were determined for six “V2-Ext-PDB6-D” derivatives. The structure of the “V2-Ext-PDB6-D” base design was determined with two different C-terminal domains, foldon and 1TD0 with a 5GS linker. In both cases, the base design appears as a perfect, closed prefusion F trimer, although it is mostly open in solution. Crystal structures were then obtained for the constructs containing the D486L/E487L (“L2”) and D486N/E487Q (“NQ”) mutations, confirming that engineered non-covalent interactions at β23 can indeed stabilize the prefusion F trimer as expected. Lastly, the crystal structure also confirmed that a well-placed inter-protomer disulfide bond can effectively lock the prefusion F in a closed trimer conformation. Second, crystal structures were determined for three “V2-Ext-PDB6-GDQ” derivatives. We focused on the constructs containing the D486N/E487Q (NQ) mutation, the inter-protomer disulfide bond, and both. Crystal structure confirmed that either mutation or a combination of both can be used to stabilize the RSV prefusion F trimer. However, a crystal structure was not obtained for the “V2-Ext-PDB6-GDQ” base. Third, crystal structures were determined for three constructs containing the disulfide bond A177C/T189C in the β3/β4 hairpin. Our crystal structure confirmed that this disulfide bond can be combined with a minimum of “V2-Ext-PDB6” base” to stabilize the RSV prefusion F trimer.

### Example 6 Optimization of the I3-01v9 Nanoparticle for Presenting Trimeric Glycoproteins with Narrow Stalks

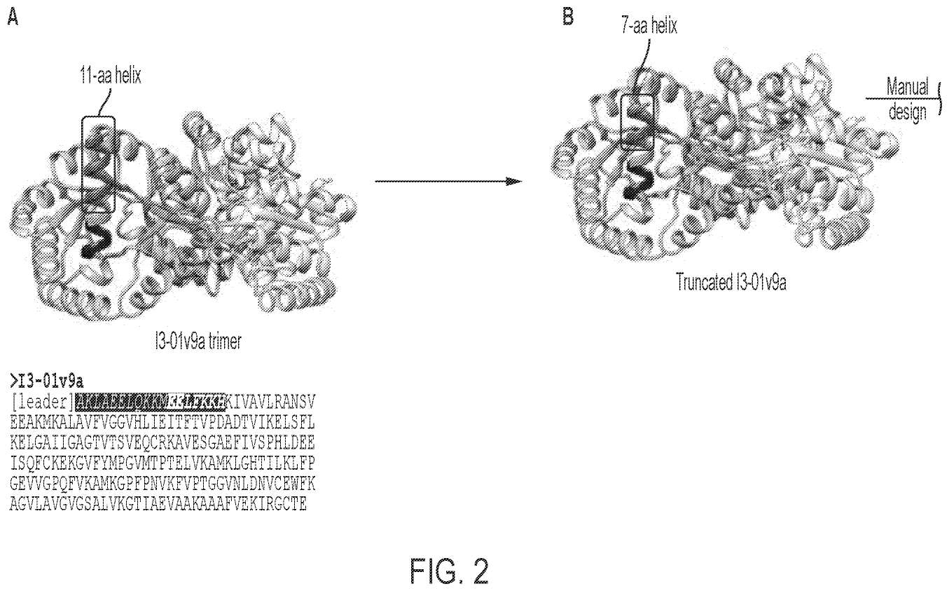

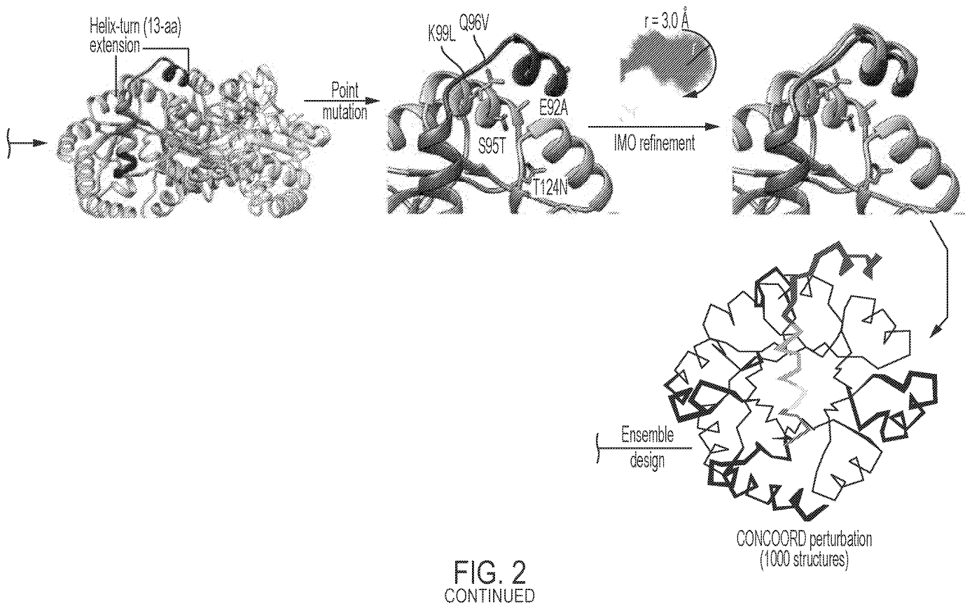

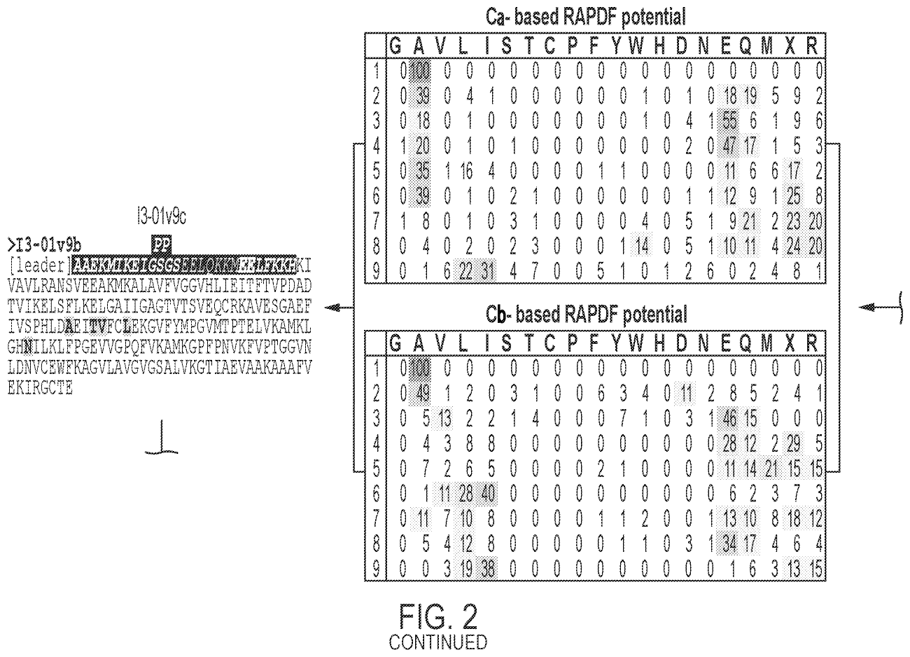

Previously, we rationally redesigned the I3-01v9 nanoparticle scaffold to optimize the display of monomeric antigens. In I3-01v9a, the N-terminal helix was extended so that its first amino acid is just above the nanoparticle surface (FIG. 2, A). Based on I3-01v9a, we further redesigned the N-terminal helix to achieve the optimal display of trimeric antigens, such as RSV prefusion F trimer (FIG. 2, B). First, the 11-aa N-terminal helix in I3-01v9a was cut to 7 aa. Then, a 13-aa helix-turn fragment (all alanine) was fused to the 7-aa helix of I3-01v9a in such a way the new N-terminal helix would pack within the groove of two helices that are part of the I3-01 core. Next, several mutations were made to the I3-01 core helices to remove steric clashes between the new N-terminal helix and the groove. A computational program named IMO (Zhu et al., Proteins 2006, 65(2):463-79) was used to relax the helix-turn backbone, which was further subjected to a protein structure sampling program, CONCOORD, to generate 1000 slightly perturbed backbone conformations. Afterwards, an ensemble-based protein design program, which was previously used to optimize HIV gp140 and HCV E2 core, was used to predict amino acids for the first 9 aa of the 13-aa fragment using Cα and Cβ-based RAPDF scoring functions (the 4-aa turn was set to be a “GSGS” (SEQ ID NO:27) linker).

We selected our final design, I3-01v9b, by combining data from predictions, and made a second design I3-01v9c by mutating the flexible turn from “GSGS” (SEQ ID NO:27) to “GPPS” (SEQ ID NO:32) to increase its rigidity. An I3-01v9b structural model was built after further backbone relaxation. The I3-01v9b N-termini form a triangle of 12.9 Å, making it suitable for displaying trimeric antigens. In a recent study, we validated the I3-01v9b/c design using the stabilized Ebola virus (EBOV) GP trimer, GPΔmuc-WL2P4, as a test case (FIG. 2, C). Briefly, the EBOV GPAmuc-WL²P⁴-I3-01v9b fusion construct was transiently expressed in HEK293F cells and purified by a mAb 100 antibody column followed by SEC. The nsEM analysis identified 2D classes corresponding to well-formed GP-I3-01v9b fusion proteins (FIG. 2, C, top). A 3D model was constructed from the EM data, showing a perfect EBOV GP trimer with a narrow stalk displayed on an I3-01v9b trimer (FIG. 2, C, bottom). Subunit sequences of the I3-01v9a, I3-01v9b and I3-01v9c nanoparticle scaffolds are shown in SEQ ID NOs: 24-26, respectively. For each of these sequences, an enzymatic site AS may be appended at the N-terminus for fusion with the antigen to be displayed. A GGGGS (SEQ ID NO:33) linker may additionally be inserted after the enzymatic site in I3-01v9a.

I3-01v9a (for monomeric antigen display)
(SEQ ID NO: 24)
AKLAEELQKKM-
EELFKKHKIVAVLRANSVEEAKMKALAVFVGGVHLIEITFTVPDADTVIKELSF
LKELGAIIGAGTVTSVEQCRKAVESGAEFIVSPHLDEEISQFCKEKGVFYMPGV
MTPTELVKAMKLGHTILKLFPGEVVGPQFVKAMKGPFPNVKFVPTGGVNLDN
VCEWFKAGVLAVGVGSALVKGTIAEVAAKAAAFVEKIRGCTE
I3-01v9b (for trimeric antigen display; with the 1^{st} residue mutated to G)
(SEQ ID NO: 25)
GAEKMIKEI-GSGS-EELQKKM-
EELFKKHKIVAVLRANSVEEAKMKALAVFVGGVHLIEITFTVPDADTVIKELSF
LKELGAIIGAGTVTSVEQCRKAVESGAEFIVSPHLDAEITVFCLEKGVFYMPGV
MTPTELVKAMKLGHNILKLFPGEVVGPQFVKAMKGPFPNVKFVPTGGVNLDN
VCEWFKAGVLAVGVGSALVKGTIAEVAAKAAAFVEKIRGCTE
I3-01v9c (for trimeric antigen display; with the 1^{st} residue mutated to G)
(SEQ ID NO: 26)
GAEKMIKEI-GPPS-EELQKKM-
EELFKKHKIVAVLRANSVEEAKMKALAVFVGGVHLIEITFTVPDADTVIKELSF
LKELGAIIGAGTVTSVEQCRKAVESGAEFIVSPHLDAEITVFCLEKGVFYMPGV
MTPTELVKAMKLGHNILKLFPGEVVGPQFVKAMKGPFPNVKFVPTGGVNLDN
VCEWFKAGVLAVGVGSALVKGTIAEVAAKAAAFVEKIRGCTE

### Example 7 Design and Negative-Stain EM Analysis of RSV Prefusion F Trimer-Presenting Nanoparticles

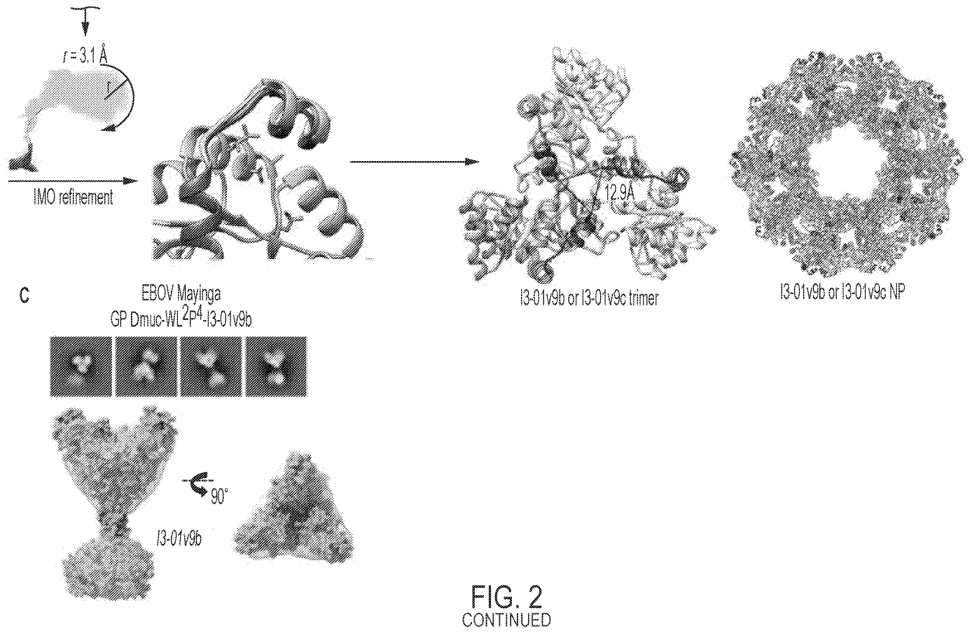

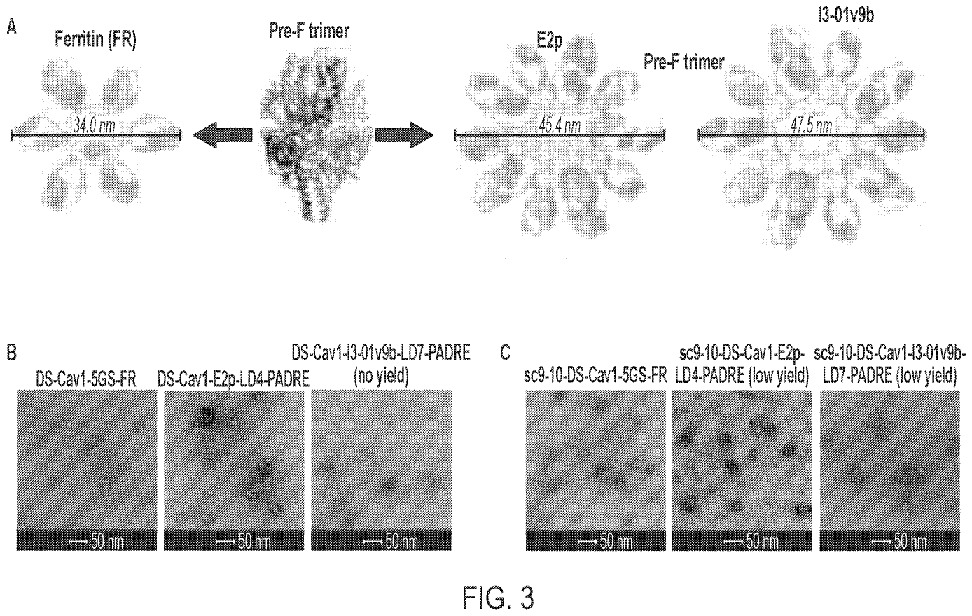

In recent studies, RSV prefusion F (DS-Cav1) was displayed on a two-component nanoparticle platform (Marcandalli et al., Cell 2019, 176(6):1420-1431.e17) and on a ferritin 24-mer (Swanson et al., Sci Immunol 2020, 5(47):eaba6466). However, our EM analysis indicates that DS-Cav1 tends to be monomeric in solution and may not be suitable for nanoparticle display. In our study, we examined the possibility of presenting known RSV prefusion F on various nanoparticle platforms and then tested our highly optimized prefusion F trimer designs for nanoparticle display. We first performed computational modeling to design RSV prefusion F trimer-presenting 1c-SApNPs (FIG. 3, A). Direct fitting of the RSV prefusion F trimer (PDB ID: 4JHW) onto the ferritin (FR) 24-mer resulted in a Ca-RMSD of 11.2 Å and produced an F-FR nanoparticle of 34 nm. Based on this calculation, a 5GS linker was added to all RSV F-FR constructs between the C-terminus of RSV F and the N-terminal residue D5 of FR. As a result, the diameter of F-5GS-FR will reach ~38 nm. Direct fitting of the RSV prefusion F trimer (PDB ID: 4JHW) onto E2p and I3-01v9b 60-mers resulted in large nanoparticles of 45.4 and 47.5 nm, respectively. As shown in our previous studies, such large trimer-presenting 1c-SApNP vaccines can induce a more potent and durable immune response than small individual antigens.

We then tested whether the DS-Cav1 prefusion F can be displayed on the three 1c-SApNP platforms (FIG. 3, B). The DS-Cav1-5GS-FR, E2p-LD4-PADRE, and I3-01v9b-LD7-PADRE constructs were transiently expressed in ExpiCHO cells and purified using a D25 antibody column. The nsEM analysis showed FR nanoparticles with irregular display of F proteins mixed with “naked” FR nanoparticles, suggestive of protein misfolding (FIG. 3, B, left). While the DS-Cav1-E2p-LD4-PADRE sample appeared to be only aggregates and debris in EM images (FIG. 3, B, middle), the DS-Cav1-I3-01v9b-LD7-PADRE construct showed extremely low yield, with no nanoparticles observed in EM images (FIG. 3, B, right). Due to the extremely low yield, SC-TM 1c-SApNP fusion constructs could not produce enough sample for nsEM analysis.

We next tested whether the sc9-10 DS-Cav1 prefusion F can be displayed on the three 1c-SApNP platforms (FIG. 3, C). Due to the presence of an inter-protomer disulfide bond, the sc9-10 DS-Cav1 1c-SApNP fusion constructs showed much lower yield than their DS-Cav1 counterparts. Nonetheless, the nsEM analysis showed FR nanoparticles displaying closed prefusion F trimers mixed with partially “naked” FR nanoparticles, suggesting that the inter-protomer disulfide bond may not form on the nanoparticle surface (FIG. 3, C, left). Both E2p and I3-01v9b constructs showed extremely low yield (FIG. 3, C, middle and right), with some partially formed particles mixed with aggregates observed for I3-01v9b (FIG. 3, C, right).

After testing the previously reported RSV prefusion F designs, we sought to test our “V2-Ext-P2DB6-D-L2/NQ” designs on FR and I3-01v9b 1c-SApNPs (FIG. 3, D). Remarkably, the EM images showed FR nanoparticles with closed prefusion F trimers for both “L2” and “NQ” constructs (FIG. 3, D, left and middle). We observed well-formed large I3-01v9b nanoparticles with prefusion F trimers evenly distributed on the surface (FIG. 3, D, right). Lastly, we tested our “V2-Ext-P2DB6-GDQ-L2/NQ” designs on FR and I3-01v9b 1c-SApNPs (FIG. 3, E). Similar success was achieved with the expression yield and structural integrity. Notably, a layer of well-formed prefusion F trimers can be seen on the surface of I3-01v9 1c-SApNPs (FIG. 3, E, right).

### Example 8 Additional Redesigned RSV Prefusion F Trimers

This Example describes additional redesigned RSV prefusion F trimer with a different minimal set of mutations to effectively stabilize the prefusion F trimer.

The sequence of the F protein of human respiratory syncytial virus A (strain A2) was obtained from GenBank with the ID (P03420). The numbering is based on the UniProt definition with the ID (P03420). Soluble F here (or termed Fd) is defined as M1-L513, with M1-G25 being the signal peptide (see SEQ ID NO:1, or A2N-WT). The uncleaved version of a soluble F was derived from A2-WT by shortening and mutating the unstructured F2 C-terminus (residues Q98-R109), by removing the 27-residue “processed active peptide” or P27 (residues E110-R136), by removing the N-terminus (residues F137-S146) of the fusion peptide (F137-V157), and by adding a 4-residue to 8-residue GS linker. Two mutations, 1379V and M447V, were added to improve F protein expression. This F construct design is considered the “base design” (SEQ ID NO: 34 and SEQ ID NO:35, or A2N-JZ0-V2-Ext and A2N-JZ0-V2-Ext2).

The uncleaved, prefusion-optimized (UFO) soluble F construct is derived from the “base design” with specific mutations incorporated into the “β3/β4 hairpin” (residues K176-S190), with the hypothesis that this region is the fundamental cause of RSV F metastability and undergoes the largest conformational change during the membrane fusion process. Two types of mutations can be introduced to the β3/β4 hairpin:

(i) Disulfide bonds between the β sheet-forming amino acids: two disulfide bond mutations—S180C/S186C and A177C/T189C—have been tested experimentally because they have the shortest C_{β}-C_{β} distances of 4.25 Å and 4.91 Å, respectively, resulting in the so-called “SS” and “AT” designs.

(ii) Mutations between the two β strands: one double mutation, S182G/N183P, has been tested experimentally because this mutation will effectively break the helical propensity at the turn between β3 and β4, resulting in the so-called “GP” design.

A total of 8 combinations (Ext vs. Ext2+SS vs. AT+no-GP vs GP) were tested:

SS-based designs: A2N-JZ0-V2-Ext-SS (SEQ ID NO:36), A2N-JZ0-V2-Ext-SSGP (SEQ ID NO:37), A2N-JZ0-V2-Ext2-SS (SEQ ID NO:38), A2N-JZ0-V2-Ext2-SSGP (SEQ ID NO:39)

AT-based designs: A2N-JZ0-V2-Ext-AT (SEQ ID NO:40), A2N-JZ0-V2-Ext-ATGP (SEQ ID NO:41), A2N-JZ0-V2-Ext2-AT (SEQ ID NO:42), A2N-JZ0-V2-Ext2-ATGP (SEQ ID NO:43)

Trimerization motifs such as foldon and viral capsid protein SHP (PDB: 1TD0) can be added to the C-terminus of a redesigned F construct with a short GS linker in between to stabilize the trimer and to increase the trimer ratio within the total protein yield. An His6-tag can be added to the C-terminus of the trimerization motif to facilitate protein purification by a Nickel column.

The C-terminus of a redesigned F construct can be fused to the N-terminus of a nanoparticle-forming subunit so that the fusion construct, when expressed in appropriate cell lines, can self-assemble into nanoparticles with prefusion F trimers displayed on the nanoparticle surface.

RSV F construct sequences (based on A2 strain wildtype sequence):

(SEQ ID NO: 2)
MELLILKANAITTILTAVTFCFASG: Leader
(SEQ ID NO: 3)
QSTPPTNNRARR: Unstructured F2 Ctm
(SEQ ID NO: 52)
ELPRFMNYTLNNAKKTNVTLSKKRKRRFLGFLLGVGSAIASGVAVSKV:
P27 peptide + fusion peptide (FP)
(SEQ ID NO: 53)
KAVVSLSNGVSVLTS: the β3/β4 hairpin region.
(A2N-JZ0-V2-Ext)
SEQ ID NO: 34
MELLILKANAITTILTAVTFCFASGQNITEEFYQSTCSAVSKGYLSALRTGWYTSVI
TIELSNIKENKCNGTDAKVKLIKQELDKYKNAVTELQLLMQSTPATGSGSAIASG
VAVSKVLHLEGEVNKIKSALLSTNKAVVSLSNGVSVLTSKVLDLKNYIDKQLLP
IVNKQSCSISNIETVIEFQQKNNRLLEITREFSVNAGVTTPVSTYMLTNSELLSLIN
DMPITNDQKKLMSNNVQIVRQQSYSIMSIIKEEVLAYVVQLPLYGVIDTPCWKL
HTSPLCTTNTKEGSNICLTRTDRGWYCDNAGSVSFFPQAETCKVQSNRVFCDT
MNSLTLPSEVNLCNVDIFNPKYDCKIMTSKTDVSSSVITSLGAIVSCYGKTKCTA
SNKNRGIIKTFSNGCDYVSNKGVDTVSVGNTLYYVNKQEGKSLYVKGEPIINFY
DPLVFPSDEFDASISQVNEKINQSLAFIRKSDELL
(A2N-JZ0-V2-Ext2)
SEQ ID NO: 35
MELLILKANAITTILTAVTFCFASGQNITEEFYQSTCSAVSKGYLSALRTGWYTSVI
TIELSNIKENKCNGTDAKVKLIKQELDKYKNAVTELQLLMQSTPATGSGSGSGS
AIASGVAVSKVLHLEGEVNKIKSALLSTNKAVVSLSNGVSVLTSKVLDLKNYIDK
QLLPIVNKQSCSISNIETVIEFQQKNNRLLEITREFSVNAGVTTPVSTYMLTNSEL
LSLINDMPITNDQKKLMSNNVQIVRQQSYSIMSIIKEEVLAYVVQLPLYGVIDTP
CWKLHTSPLCTTNTKEGSNICLTRTDRGWYCDNAGSVSFFPQAETCKVQSNRV
FCDTMNSLTLPSEVNLCNVDIFNPKYDCKIMTSKTDVSSSVITSLGAIVSCYGKT
KCTASNKNRGIIKTFSNGCDYVSNKGVDTVSVGNTLYYVNKQEGKSLYVKGEP
IINFYDPLVFPSDEFDASISQVNEKINQSLAFIRKSDELL
(A2N-JZ0-V2-Ext-SS)
SEQ ID NO: 36
MELLILKANAITTILTAVTFCFASGQNITEEFYQSTCSAVSKGYLSALRTGWYTSVI
TIELSNIKENKCNGTDAKVKLIKQELDKYKNAVTELQLLMQSTPATGSGSAIASG
VAVSKVLHLEGEVNKIKSALLSTNKAVVCLSNGVCVLTSKVLDLKNYIDKQLL
PIVNKQSCSISNIETVIEFQQKNNRLLEITREFSVNAGVTTPVSTYMLTNSELLSLI
NDMPITNDQKKLMSNNVQIVRQQSYSIMSIIKEEVLAYVVQLPLYGVIDTPCWK
LHTSPLCTTNTKEGSNICLTRTDRGWYCDNAGSVSFFPQAETCKVQSNRVFCD
TMNSLTLPSEVNLCNVDIFNPKYDCKIMTSKTDVSSSVITSLGAIVSCYGKTKCT
ASNKNRGIIKTFSNGCDYVSNKGVDTVSVGNTLYYVNKQEGKSLYVKGEPIINF
YDPLVFPSDEFDASISQVNEKINQSLAFIRKSDELL
(A2N-JZ0-V2-Ext-SSGP)
SEQ ID NO: 37
MELLILKANAITTILTAVTFCFASGQNITEEFYQSTCSAVSKGYLSALRTGWYTSVI
TIELSNIKENKCNGTDAKVKLIKQELDKYKNAVTELQLLMQSTPATGSGSAIASG
VAVSKVLHLEGEVNKIKSALLSTNKAVVCLGPGVCVLTSKVLDLKNYIDKQLL
PIVNKQSCSISNIETVIEFQQKNNRLLEITREFSVNAGVTTPVSTYMLTNSELLSLI
NDMPITNDQKKLMSNNVQIVRQQSYSIMSIIKEEVLAYVVQLPLYGVIDTPCWK
LHTSPLCTTNTKEGSNICLTRTDRGWYCDNAGSVSFFPQAETCKVQSNRVFCD
TMNSLTLPSEVNLCNVDIFNPKYDCKIMTSKTDVSSSVITSLGAIVSCYGKTKCT
ASNKNRGIIKTFSNGCDYVSNKGVDTVSVGNTLYYVNKQEGKSLYVKGEPIINF
YDPLVFPSDEFDASISQVNEKINQSLAFIRKSDELL
(A2N-JZ0-V2-Ext2-SS)
SEQ ID NO: 38
MELLILKANAITTILTAVTFCFASGQNITEEFYQSTCSAVSKGYLSALRTGWYTSVI
TIELSNIKENKCNGTDAKVKLIKQELDKYKNAVTELQLLMQSTPATGSGSGSGS
AIASGVAVSKVLHLEGEVNKIKSALLSTNKAVVCLSNGVCVLTSKVLDLKNYID
KQLLPIVNKQSCSISNIETVIEFQQKNNRLLEITREFSVNAGVTTPVSTYMLINSE
LLSLINDMPITNDQKKLMSNNVQIVRQQSYSIMSIIKEEVLAYVVQLPLYGVIDT
PCWKLHTSPLCTTNTKEGSNICLTRTDRGWYCDNAGSVSFFPQAETCKVQSNR
VFCDTMNSLTLPSEVNLCNVDIFNPKYDCKIMTSKTDVSSSVITSLGAIVSCYGK
TKCTASNKNRGIIKTFSNGCDYVSNKGVDTVSVGNTLYYVNKQEGKSLYVKG
EPIINFYDPLVFPSDEFDASISQVNEKINQSLAFIRKSDELL
(A2N-JZ0-V2-Ext2-SSGP)
SEQ ID NO: 39
MELLILKANAITTILTAVTFCFASGQNITEEFYQSTCSAVSKGYLSALRTGWYTSVI
TIELSNIKENKCNGTDAKVKLIKQELDKYKNAVTELQLLMQSTPATGSGSGSGS
AIASGVAVSKVLHLEGEVNKIKSALLSTNKAVVCLGPGVCVLTSKVLDLKNYID
KQLLPIVNKQSCSISNIETVIEFQQKNNRLLEITREFSVNAGVTTPVSTYMLINSE
LLSLINDMPITNDQKKLMSNNVQIVRQQSYSIMSIIKEEVLAYVVQLPLYGVIDT
PCWKLHTSPLCTTNTKEGSNICLTRTDRGWYCDNAGSVSFFPQAETCKVQSNR
VFCDTMNSLTLPSEVNLCNVDIFNPKYDCKIMTSKTDVSSSVITSLGAIVSCYGK
TKCTASNKNRGIIKTFSNGCDYVSNKGVDTVSVGNTLYYVNKQEGKSLYVKG
EPIINFYDPLVFPSDEFDASISQVNEKINQSLAFIRKSDELL
(A2N-JZ0-V2-Ext-AT)
SEQ ID NO: 40
MELLILKANAITTILTAVTFCFASGQNITEEFYQSTCSAVSKGYLSALRTGWYTSVI
TIELSNIKENKCNGTDAKVKLIKQELDKYKNAVTELQLLMQSTPATGSGSAIASG
VAVSKVLHLEGEVNKIKSALLSTNKCVVSLSNGVSVLCSKVLDLKNYIDKQLLP
IVNKQSCSISNIETVIEFQQKNNRLLEITREFSVNAGVTTPVSTYMLTNSELLSLIN
DMPITNDQKKLMSNNVQIVRQQSYSIMSIIKEEVLAYVVQLPLYGVIDTPCWKL
HTSPLCTTNTKEGSNICLTRTDRGWYCDNAGSVSFFPQAETCKVQSNRVFCDT
MNSLTLPSEVNLCNVDIFNPKYDCKIMTSKTDVSSSVITSLGAIVSCYGKTKCTA
SNKNRGIIKTFSNGCDYVSNKGVDTVSVGNTLYYVNKQEGKSLYVKGEPIINFY
DPLVFPSDEFDASISQVNEKINQSLAFIRKSDELL
(A2N-JZ0-V2-Ext-ATGP)
SEQ ID NO: 41
MELLILKANAITTILTAVTFCFASGQNITEEFYQSTCSAVSKGYLSALRTGWYTSVI
TIELSNIKENKCNGTDAKVKLIKQELDKYKNAVTELQLLMQSTPATGSGSAIASG
VAVSKVLHLEGEVNKIKSALLSTNKCVVSLGPGVSVLCSKVLDLKNYIDKQLLP
IVNKQSCSISNIETVIEFQQKNNRLLEITREFSVNAGVTTPVSTYMLTNSELLSLIN
DMPITNDQKKLMSNNVQIVRQQSYSIMSIIKEEVLAYVVQLPLYGVIDTPCWKL
HTSPLCTTNTKEGSNICLTRTDRGWYCDNAGSVSFFPQAETCKVQSNRVFCDT
MNSLTLPSEVNLCNVDIFNPKYDCKIMTSKTDVSSSVITSLGAIVSCYGKTKCTA
SNKNRGIIKTFSNGCDYVSNKGVDTVSVGNTLYYVNKQEGKSLYVKGEPIINFY
DPLVFPSDEFDASISQVNEKINQSLAFIRKSDELL
(A2N-JZ0-V2-Ext2-AT)
SEQ ID NO: 42
MELLILKANAITTILTAVTFCFASGQNITEEFYQSTCSAVSKGYLSALRTGWYTSVI
TIELSNIKENKCNGTDAKVKLIKQELDKYKNAVTELQLLMQSTPATGSGSGSGS
AIASGVAVSKVLHLEGEVNKIKSALLSTNKCVVSLSNGVSVLCSKVLDLKNYID
KQLLPIVNKQSCSISNIETVIEFQQKNNRLLEITREFSVNAGVTTPVSTYMLINSE
LLSLINDMPITNDQKKLMSNNVQIVRQQSYSIMSIIKEEVLAYVVQLPLYGVIDT
PCWKLHTSPLCTTNTKEGSNICLTRTDRGWYCDNAGSVSFFPQAETCKVQSNR
VFCDTMNSLTLPSEVNLCNVDIFNPKYDCKIMTSKTDVSSSVITSLGAIVSCYGK
TKCTASNKNRGIIKTFSNGCDYVSNKGVDTVSVGNTLYYVNKQEGKSLYVKG
EPIINFYDPLVFPSDEFDASISQVNEKINQSLAFIRKSDELL
(A2N-JZ0-V2-Ext2-ATGP)
SEQ ID NO: 43
MELLILKANAITTILTAVTFCFASGQNITEEFYQSTCSAVSKGYLSALRTGWYTSVI
TIELSNIKENKCNGTDAKVKLIKQELDKYKNAVTELQLLMQSTPATGSGSGSGS
AIASGVAVSKVLHLEGEVNKIKSALLSTNKCVVSLGPGVSVLCSKVLDLKNYID
KQLLPIVNKQSCSISNIETVIEFQQKNNRLLEITREFSVNAGVTTPVSTYMLINSE
LLSLINDMPITNDQKKLMSNNVQIVRQQSYSIMSIIKEEVLAYVVQLPLYGVIDT
PCWKLHTSPLCTTNTKEGSNICLTRTDRGWYCDNAGSVSFFPQAETCKVQSNR
VFCDTMNSLTLPSEVNLCNVDIFNPKYDCKIMTSKTDVSSSVITSLGAIVSCYGK
TKCTASNKNRGIIKTFSNGCDYVSNKGVDTVSVGNTLYYVNKQEGKSLYVKG
EPIINFYDPLVFPSDEFDASISQVNEKINQSLAFIRKSDELL

### Example 9 Vaccine Antigen Expression and Purification

This Example describes expression and purification of redesigned prefusion F trimer vaccine antigens as described in Example 8.

Cell line: All F trimers were expressed in HEK293F/Expi293F cells and ExpiCHO cells, with ExpiCHO showing greater protein yield. All F-presenting nanoparticles were expressed in ExpiCHO cells.

Purification: After transient expression, RSV F-containing antigens were purified from the supernatant using antigen-specific antibody columns based on (1) a prefusion site-Ø-specific neutralizing antibody D25 and (2) a neutralizing antibody MPE8 that recognizes two protomers of the F trimer. MPE8 binds to both prefusion and postfusion F trimers but favors the prefusion structure. Both D25 and MPE8 antibody columns can effectively purify RSV F trimers and nanoparticles, with D25 showing a greater protein yield. For hMPV and PIV1-5 F trimers, a Nickel column is used for His-tag purification. Antibody columns will be developed for tag-free trimer/nanoparticle purification.

### Example 10 Study of Paramyxovirus F Metastability with Known Prefusion F Trimers

This Example describes characterization of prefusion F trimer that have been reported in the literature.

We set out to investigate the fundamental cause of paramyxovirus F metastability and to develop a simple, general, and effective prefusion F trimer stabilization strategy that can be applied to all members of the paramyxovirus family and enable the multivalent display of stabilized F trimers on self-assembling nanoparticles as virus-like particle (VLP) vaccines. Since RSV has been most studied in structure-based vaccine design with three representative prefusion F designs available, RSV was chosen as a focus of the current study with hMPV and PIV3 included to confirm our F stabilization strategy. We first characterized three RSV F designs that have been reported in the literature, DS-Cav1 (McLellan et al., Science 342:592-8, 2013), sc9-10 DS-Cav1 (Joyce et al., Nat Struct Mol Biol 23:811-820, 2016), and SC-TM (Krarup et al., Nat Commun 6:8143, 2015), to compare their expression, trimer formation, and purification.

In terms of expression, the effect of cell lines on the yield and purity of RSV F trimers was examined using HEK293F, a transient mammalian cell line, and ExpiCHO, a transient high-yield version of the industrial CHO-S cell line. In terms of purification method, two immunoaffinity columns based on D25, which recognizes the prefusion site-Ø, and MPE8, which binds two adjacent F subunits, were evaluated for their ability as antibody columns for tag-free purification. In terms of trimer stability, the effect of C-terminal trimerization motif was examined using a soluble F construct without C-terminal motif and two F constructs with foldon and 1TD0 motifs at their C-termini.

Three representative RSV prefusion F constructs, DS-Cav1, sc9-10 DS-Cav1, and SC-TM, were expressed transiently in HEK 293F and ExpiCHO cells. Our previous study (He et al., Sci Adv 4(11):eaau6769, 2018) showed that HIV-1 gp140 trimers can be expressed in ExpiCHO cells with significantly greater yield and purity than in 293F cells. Based on this finding, a small volume of 25 ml ExpiCHO cells was used for transfection followed by purification using D25 and MPE8 antibody columns, whereas 400 ml and 100 ml 293F cells were used for transection, which was followed by D25 and MPE8 purification, respectively. Since D25 recognizes site Ø of the prefusion F regardless of F being in a monomeric or multimeric state (e.g., dimer, trimer, and aggregate), D25 was expected to give higher overall F protein (as well as trimer) yield than MPE8 and provide a more complete profile. Therefore, a higher volume was used in the evaluation of the D25 antibody column in combination with 293F cells to maximize the usefulness of the data obtained from this combination. Following transient expression and antibody purification, RSV F protein was characterized by size-exclusion chromatography (SEC) using a Superdex 200 10/300 column.

The three RSV prefusion F trimers demonstrated distinct design-specific patterns. Overall, the optimized sc9-10 DS-Cav1 design significantly outperformed the original DS-Cav1 design and the SC-TM design in almost every aspect examined in this comparison. Of particular note, sc9-10 DS-Cav1 showed the highest trimer yield and purity when it was fused with a trimerization motif at C-terminus, but produced primarily monomers without any C-terminal trimerization motifs, suggesting that the fundamental cause of F metastability is still present in sc9-10 DS-Cav1. In terms of the effect of cell lines on F expression, DS-Cav1 and SC-TM showed little to no yield in 293F cells but behaved differently in ExpiCHO cells, in which DS-Cav1 showed a measurable level of expression whereas SC-TM did not. Therefore, the three prefusion F design constructs can be ranked as sc9-10 DS-Cav1>>DS-Cav1>>SC-TM in terms of protein expression, with ExpiCHO being a more suitable expression system.

In terms of antibody columns, DS-Cav1 and sc9-10 DS-Cav1 exhibited rather different patterns: for DS-Cav1, D25 showed somewhat lower yield than MPE8 for constructs without trimerization motif and with foldon, whereas for sc9-10 DS-CaV1, D25 gave significantly higher yield than MPE8 as indicated by the UV280 absorbance value in SEC profiles.

In terms of trimer stability, it is evident from the SEC profiles that both DS-Cav1 and sc9-10 DS-CaV1 require a C-terminal trimerization motif to remain trimeric, suggesting that these F designs do not improve trimer formation or the major cause of F metastability is still present in these constructs. Finally, in terms of the composition of F protein, DS-Cav1 and sc9-10 DS-Cav1 also exhibited different patterns: DS-Cav1 appeared to have a higher-molecular-weight peak at 11 ml, which was merged with the trimer peak at 12 ml when foldon was used, but this the peak of this F species could be clearly seen for another C-terminal trimerization motif, 1TD0; in contrast, sc9-10 DS-Cav1 showed a single trimer peak at 12 mL when foldon was used but produced aggregates when 1TD0 was used. It is also worth noting that 1TD0 produced a narrower trimer peak than that produced by foldon, suggesting a greater trimer purity for the 1TD0-attached sc9-10 DS-Cav1 F trimer. Blue native polyacrylamide gel electrophoresis (BN-PAGE) demonstrated consistent monomer and trimer bands for different F constructs.

### Example 11 Characterization of Redesigned RSV F Trimers

This Example describes characterization of our redesigned RSV prefusion F trimer vaccine antigens as described in Example 8.

Based on our hypothesis that the β3/β4 hairpin is the fundamental cause of RSV F metastability, we investigated two disulfide bond mutations to lock this region in its prefusion structure—a β-hairpin—and to prevent its transition to the postfusion structure—an extended α-helix. These two disulfide bond mutants are termed A2N-JZ0-V2-Ext-SS and A2N-JZ0-V2-Ext-AT or simply V2-Ext-SS and V2-Ext-AT (SEQ ID NO: 36 and SEQ ID NO:40). These three newly designed F constructs were characterized using the previously described protocol. Overall, both disulfide bond mutants outperformed the base design, with V2-Ext-AT being the best performer, which produced a significant trimer peak of high yield without any C-terminal trimerization motif. These results provide the most convincing evidence that the β3/β4 hairpin is the fundamental cause of RSV F metastability and that as minimum as a single disulfide bond mutation to this region, if properly introduced, can effectively eliminate the metastability. The base design could be expressed in both cell lines, with greater yield obtained from the ExpiCHO cells.

In terms of antibody columns, D25 consistently gave higher yield than MPB8. It is worth pointing out that MPE8, which recognizes two F protomers of the trimer, did not show a preference for trimer over other species in the produced F protein. For example, a more pronounced trimer peak was observed for V2-Ext without any C-terminal trimerization motif that was expressed in ExpiCHO cells after D25 purification. In terms of trimer formation, a trimerization motif fused to the C-terminus of V2-Ext may result in more difficult trimer folding, e.g., no yield for the V2-Ext-foldon construct, or aggregation, e.g., a peak corresponding to high-molecular-weight aggregates (8-10 mL) in ExpiCHO cells for the V2-Ext-1TD0 construct. The first mutant (V2-Ext-SS), with a disulfide bond engineered near the β-turn (one residue away), showed significantly increased F protein expression in both cell lines, with ExpiCHO slightly outperforming HEK 293F. In terms of antibody column, D25 and MPE8 yielded similar SEC profiles for ExpiCHO-produced proteins but behaved differently for 293F-produced proteins.

In addition, this disulfide bond mutation appeared to be more effective at trimer stabilization when used with foldon than with 1TD0, or than without any C-terminal trimerization motif. The second mutant (V2-Ext-AT), with a disulfide bond engineered at the distal end of the β3/β4 hairpin, showed the most desirable properties, although its yield is lower than that of the first disulfide bond mutant. In addition to yield, the two disulfide bond mutations differ notably in their ability to facilitate trimer folding and in their compatibility with different C-terminal trimerization motifs. Specifically, AT appeared to be much more effective than SS in promoting trimer formation when no C-terminal trimerization motif was attached. The AT disulfide bond design also appeared to be more compatible with 1TD0 than with foldon.

We next investigated whether adding a double mutation (GP) to the two disulfide bond mutants at the β-turn will further destabilize the postfusion helix and, as a result, stabilize the prefusion β3/β4 hairpin. Surprisingly, the GP mutation at the β-turn exerted drastically different effects on the two disulfide bond mutants. The SSGP design showed much improved trimer ratio in the produced F protein, which was accompanied by a notable reduction of total yield. A higher trimer peak was also observed for the V2-Ext-SSGP constructs fused with foldon and 1TD0, irrespective of the cell line used. In contrast, GP produced a primarily negative impact on the AT-containing F constructs. Although the ATGP design showed lower monomer and aggregate ratios, the overall yield appeared to be too low for vaccine production in CHO cells. In summary, V2-Ext-SSGP provides a promising alternative to V2-Ext-AT for prefusion F trimer design.

Finally, we investigated whether a longer linker between F2 and F1 may improve the two “best” prefusion F constructs identified thus far—V2-Ext-SSGP and V2-Ext-AT. Remarkably, the Ext2 mutation consistently improved the trimer ratio without any C-terminal trimerization motif. However, the use of a long cleavage-site linker appeared to have a negative effect on any F construct with a C-terminal trimerization motif, in terms of either trimer ratio or trimer yield. In summary, a long cleavage site linker may be used without a trimerization motif.

### Example 12 Redesigned hMPV and PIV Prefusion F Trimers

This Example describes redesigned hMPV and PIV prefusion F trimers, with a minimal set of mutations corresponding to that used for RSV F trimers described in Example 8, to effectively stabilize the prefusion F trimer. More details of the studies are described in Example 13.

The sequence of the F protein of human metapneumovirus hMPV (isolate “TN03.03.19”) is obtained from GenBank with the ID AEZ52364. The numbering is based on the crystal structure (PDB ID: 5WB0) and the UniProt definition for another hMPV strain (strain CAN97-83) with the ID Q6WB98. Soluble F is defined as M1-T489, with M1-G18 being the signal peptide (see SEQ ID NO:44, or TN-WT). A shortened version of soluble F (equivalent to Fd for hRSV) is defined as M1-L481, with M1-G18 being the signal peptide (see SEQ ID NO:45, or TN-WT-cut). The uncleaved prefusion-optimized (UFO) soluble F construct was based on the TN-WT-cut design with specific mutations incorporated into the equivalent “β3/β4 hairpin” (residues E146-T160) based on the hypothesis that this region is the fundamental cause of hMPV F metastability and undergoes the largest conformational change during the membrane fusion process. Disulfide bond(s) between the β sheet-forming amino acids can be introduced to lock the hMPV F structure in the prefusion state. Mutations that introduce a disulfide bond, A147C/A159C, were tested with two different ways of dealing with the fusion peptide (FP) (see SEQ ID NO:46 and SEQ ID NO:47, or TN-cut-UFO1 and TN-cut-UFO2) to validate the metastability hypothesis and the importance of the “β3/β4 hairpin” to hMPV F.

Trimerization motifs such as foldon and viral capsid protein SHP (PDB: 1TD0) can be added to the C-terminus of a redesigned F construct with a short GS linker in between to stabilize the trimer and to increase the trimer ratio within the total protein yield. An His6-tag can be added to the C-terminus of the trimerization motif to facilitate protein purification by a Nickel column.

The C-terminus of a redesigned F construct can be fused to the N-terminus of a nanoparticle-forming subunit so that the fusion construct, when expressed in appropriate cell lines, can self-assemble into nanoparticles with prefusion F trimers displayed on the nanoparticle surface.

hMPV F Construct Sequences:

(SEQ ID NO: 54)
MSWKVVIIFSLLITPQHG: leader
(SEQ ID NO: 55)
DQLAREEQIENPRQSRFVLGAIALGV: unstructured F2 N-terminus +
cleavage site + fusion peptide
(SEQ ID NO: 56)
EAVSTLGNGVRVLAT the equivalent β3/β4 hairpin region
(TN-WT)
SEQ ID NO: 44
MSWKVVIIFSLLITPQHGLKESYLEESCSTITEGYLSVLRTGWYTNVFTLEVGDVE
NLTCADGPSLIKTELDLTKSALRELRTVSADQLAREEQIENPRQSRFVLGAIALG
VATAAAVTAGVAIAKTIRLESEVTAIKNALKKTNEAVSTLGNGVRVLATAVRE
LKDFVSKNLTRAINKNKCDIADLKMAVSFSQFNRRFLNVVRQFSDNAGITPAIS
LDLMTDAELARAVSNMPTSAGQIKLMLENRAMVRRKGFGILIGVYGSSVIYMV
QLPIFGVIDTPCWIVKAAPSCSEKKGNYACLLREDQGWYCQNAGSTVYYPNEK
DCETRGDHVFCDTAAGINVAEQSKECNINISTTNYPCKVSTGRHPISMVALSPL
GALVACYKGVSCSIGSNRVGIIKQLNKGCSYITNQDADTVTIDNTVYQLSKVEG
EQHVIKGRPVSSSFDPVKFPEDQFNVALDQVFESIENSQALVDQSNRILSSAEKG
NT
(TN-WT-cut)
SEQ ID NO: 45
MSWKVVIIFSLLITPQHGLKESYLEESCSTITEGYLSVLRTGWYTNVFTLEVGDVE
NLTCADGPSLIKTELDLTKSALRELRTVSADQLAREEQIENPRQSRFVLGAIALG
VATAAAVTAGVAIAKTIRLESEVTAIKNALKKTNEAVSTLGNGVRVLATAVRE
LKDFVSKNLTRAINKNKCDIADLKMAVSFSQFNRRFLNVVRQFSDNAGITPAIS
LDLMTDAELARAVSNMPTSAGQIKLMLENRAMVRRKGFGILIGVYGSSVIYMV
QLPIFGVIDTPCWIVKAAPSCSEKKGNYACLLREDQGWYCQNAGSTVYYPNEK
DCETRGDHVFCDTAAGINVAEQSKECNINISTTNYPCKVSTGRHPISMVALSPL
GALVACYKGVSCSIGSNRVGIIKQLNKGCSYITNQDADTVTIDNTVYQLSKVEG
EQHVIKGRPVSSSFDPVKFPEDQFNVALDQVFESIENSQALVDQSNRIL
(TN-cut-UFO1)
SEQ ID NO: 46
MSWKVVIIFSLLITPQHGLKESYLEESCSTITEGYLSVLRTGWYTNVFTLEVGDVE
NLTCADGPSLIKTELDLTKSALRELRTVSAGSGSGSGSFVLGAIALGVATAAAV
TAGVAIAKTIRLESEVTAIKNALKKTNECVSTLGNGVRVLCTAVRELKDFVSK
NLTRAINKNKCDIADLKMAVSFSQFNRRFLNVVRQFSDNAGITPAISLDLMTDA
ELARAVSNMPTSAGQIKLMLENRAMVRRKGFGILIGVYGSSVIYMVQLPIFGVI
DTPCWIVKAAPSCSEKKGNYACLLREDQGWYCQNAGSTVYYPNEKDCETRGD
HVFCDTAAGINVAEQSKECNINISTTNYPCKVSTGRHPISMVALSPLGALVACY
KGVSCSIGSNRVGIIKQLNKGCSYITNQDADTVTIDNTVYQLSKVEGEQHVIKG
RPVSSSFDPVKFPEDQFNVALDQVFESIENSQALVDQSNRIL
(TN-cut-UFO2)
SEQ ID NO: 47
MSWKVVIIFSLLITPQHGLKESYLEESCSTITEGYLSVLRTGWYTNVFTLEVGDVE
NLTCADGPSLIKTELDLTKSALRELRTVSAGSGSGSGSVATAAAVTAGVAIAKT
IRLESEVTAIKNALKKTNECVSTLGNGVRVLCTAVRELKDFVSKNLTRAINKN
KCDIADLKMAVSFSQFNRRFLNVVRQFSDNAGITPAISLDLMTDAELARAVSN
MPTSAGQIKLMLENRAMVRRKGFGILIGVYGSSVIYMVQLPIFGVIDTPCWIVK
AAPSCSEKKGNYACLLREDQGWYCQNAGSTVYYPNEKDCETRGDHVFCDTA
AGINVAEQSKECNINISTTNYPCKVSTGRHPISMVALSPLGALVACYKGVSCSIG
SNRVGIIKQLNKGCSYITNQDADTVTIDNTVYQLSKVEGEQHVIKGRPVSSSFDP
VKFPEDQFNVALDQVFESIENSQALVDQSNRIL

PIV3 F: The sequence of the F protein of human parainfluenza virus type 3 (PIV3) (strain “HPIV3/USA/629-D01959/2007”) is obtained from GenBank with the ID (AGW51052). The numbering is based on the cryo-EM structure (PDB ID: 6MJZ) and the UniProt definition for a recombinant PIV3/PIV1 virus with the ID (055888). Soluble F sequence (equivalent to Fd for hRSV) is defined as M1-T484, with M1-C₁₈ being the signal peptide (see SEQ ID NO:48, or PIV3-WT).

The uncleaved prefusion-optimized (UFO) soluble F construct is based on the PIV3-WT design after removal of the unstructured F2 N-terminus and cleavage site and with specific mutations incorporated into the “β1/β2 hairpin” (residues V158-I172) based on the hypothesis that this region is the fundamental cause of PIV3 F metastability and undergoes the largest conformational change during the membrane fusion process. Disulfide bonds between the β sheet-forming amino acids in the strands V158-V161 and I169-I172 can be introduced to lock the PIV3 F structure in the prefusion state. It must be noted that any disulfide bonds introduced within Q162-L168 may cause structural distortions. Mutations that introduce a disulfide bond, Q159C/A171C, have been tested with two different ways of dealing with the fusion peptide (FP) region (see SEQ ID NO:61 and SEQ ID NO:62, or PIV3-UFO1 and PIV3-UFO2) to validate the metastability hypothesis and the importance of the “β1/β2 hairpin” to PIV3 F.

Trimerization motifs such as foldon and viral capsid protein SHP (PDB: 1TD0) can be added to the C-terminus of a redesigned F construct with a short GS linker in between to stabilize the trimer and to increase the trimer ratio within the total protein yield. An His6-tag can be added to the C-terminus of the trimerization motif to facilitate protein purification on a Nickel column.

The C-terminus of a redesigned F construct can be fused to the N-terminus of a nanoparticle-forming subunit so that the fusion construct, when expressed in appropriate cell lines, can self-assemble into nanoparticles with prefusion F trimers displayed on the nanoparticle surface.

Other PIVs: given the high structural similarity between PIV3, PIV5, and other PIVs, the UFO trimer constructs can be designed following the same principles to modify the “β1/β2 hairpin region.

### PIV3 F Construct Sequences:

(SEQ ID NO: 57)
MLISILSIITTMIMASHC: leader
(SEQ ID NO: 58)
GLKLQKDVIVTNOESNENTDPRTERFFGGVIGTIALGV:
unstructured F2 N-terminus + cleavage site + fusion peptide
(SEQ ID NO: 59)
VQSVQSSVGNLIVAI: β1/β2 hairpin, equivalent to the β3/β4 hairpin
region.
(PIV3-WT)
SEQ ID NO: 48
MLISILSIITTMIMASHCQIDITKLQHVGVLVNSPKGMKISQNFETRYLILSLIPKIED
SNSCGDQQIKQYKRLLDRLIIPLYDGLKLQKDVIVTNQESNENTDPRTERFFGGV
IGTIALGVATSAQITAAVALVEAKQAKSDIEKLKEAIRDTNKAVQSVQSSVGN
LIVAIKSVQDYVNKEIVPSIARLGCEAAGLQLGIALTQHYSELTNIFGDNIGSLQE
KGIKLQGIASLYRTNITEIFTTSTVDKYDIYDLLFTESIKVRVIDVDLNDYSITLQV
RLPLLTRLLNTQIYKVDSISYNIQNREWYIPLPSHIMTKGAFLGGADVKECIEAF
SSYICPSDPGFVLNHEMESCLSGNISQCPRTTVTSDIVPRYAFVNGGVVANCITT
TCTCNGIGNRINQPPDQGVKIITHKECNTIGINGMLFNTNKEGTLAFYTPDDITL
NNSVALDPIDISIELNKAKSDLEESKEWIRRSNQKLDSI
(PIV3-UFO1)
SEQ ID NO: 61
MLISILSIITTMIMASHCQIDITKLQHVGVLVNSPKGMKISQNFETRYLILSLIPKIED
SNSCGDQQIKQYKRLLDRLIIPLYDGLKLQKDVIVTGSGSFFGGVIGTIALGVA
TSAQITAAVALVEAKQAKSDIEKLKEAIRDTNKAVCSVQSSVGNLIVCIKSVQD
YVNKEIVPSIARLGCEAAGLQLGIALTQHYSELTNIFGDNIGSLQEKGIKLQGIAS
LYRTNITEIFTTSTVDKYDIYDLLFTESIKVRVIDVDLNDYSITLQVRLPLLTRLL
NTQIYKVDSISYNIQNREWYIPLPSHIMTKGAFLGGADVKECIEAFSSYICPSDPG
FVLNHEMESCLSGNISQCPRTTVTSDIVPRYAFVNGGVVANCITTTCTCNGIGN
RINQPPDQGVKIITHKECNTIGINGMLFNTNKEGTLAFYTPDDITLNNSVALDPID
ISIELNKAKSDLEESKEWIRRSNQKLDSI
(PIV3-UFO2)
SEQ ID NO: 62
MLISILSIITTMIMASHCQIDITKLQHVGVLVNSPKGMKISQNFETRYLILSLIPKIED
SNSCGDQQIKQYKRLLDRLIIPLYDGLKLQKDVGSGSATSAQITAAVALVEAKQ
AKSDIEKLKEAIRDTNKAVCSVQSSVGNLIVCIKSVQDYVNKEIVPSIARLGCEA
AGLQLGIALTQHYSELTNIFGDNIGSLQEKGIKLQGIASLYRTNITEIFTTSTVDK
YDIYDLLFTESIKVRVIDVDLNDYSITLQVRLPLLTRLLNTQIYKVDSISYNIQNR
EWYIPLPSHIMTKGAFLGGADVKECIEAFSSYICPSDPGFVLNHEMESCLSGNIS
QCPRTTVTSDIVPRYAFVNGGVVANCITTTCTCNGIGNRINQPPDQGVKIITHKE
CNTIGINGMLFNTNKEGTLAFYTPDDITLNNSVALDPIDISIELNKAKSDLEESKE
WIRRSNQKLDSI

### Example 13 Characterization of Redesigned F Trimers of Other Paramyxoviruses

Having established that the β3/β4 hairpin is the fundamental cause of RSV F metastability as described in Examples 8-11, we examined the possibility of extending this design concept to other members of the paramyxovirus family. To examine this possibility, we generated two sets of UFO constructs for hMPV F (SEQ ID NO: 46 and SEQ ID NO:47) and PIV3 F (SEQ ID NO:61 and SEQ ID NO:62), all having a C-terminal trimerization motif (1TD0) followed by a His6-tag.

In preliminary tests, all four constructs were expressed transiently in 250 mL 293F cells and purified using a Nickel column prior to SEC. Overall, the UFO2 design notably outperformed the UFO1 design, which contained the fusion peptide (FP), for both hMPV and PIV3. For hMPV, a significant trimer peak (11~12 mL) was observed for UFO2 but not UFO1, consistent with the BN-PAGE analysis. For PIV3, the UFO2 construct produced a high trimer peak (11~12 mL) with a notable aggregate peak (8-10 mL), whereas the UFO1 construct produced primarily aggregates in 293F cells, similar to our observation for the hMPV constructs.

In summary, this comparative analysis confirms the notation that the β3/β4 and β1/β2 hairpins are the fundamental causes of F metastability for hMPV and PIV3, respectively. Considering the similarity among PIV1-5, this result also suggests that the UFO2 design can also be applied to other PIVs.

### Example 14 EM Characterization of Nanoparticles Displaying RSV F Trimers

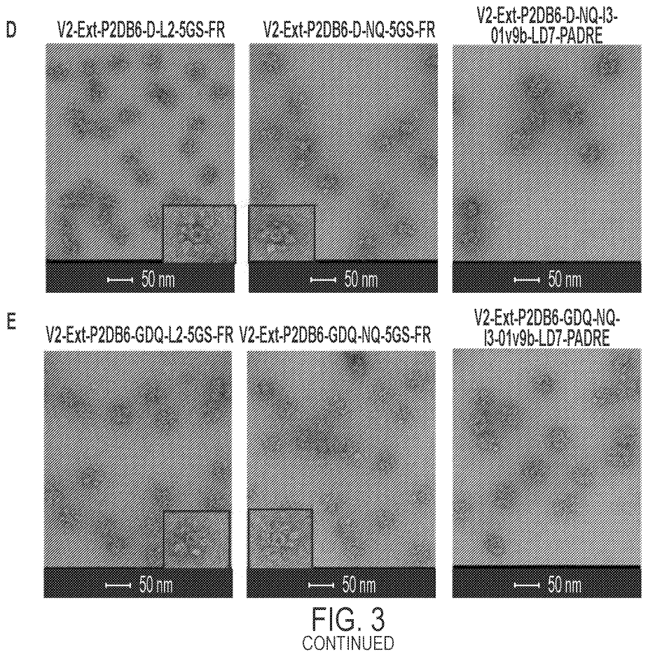

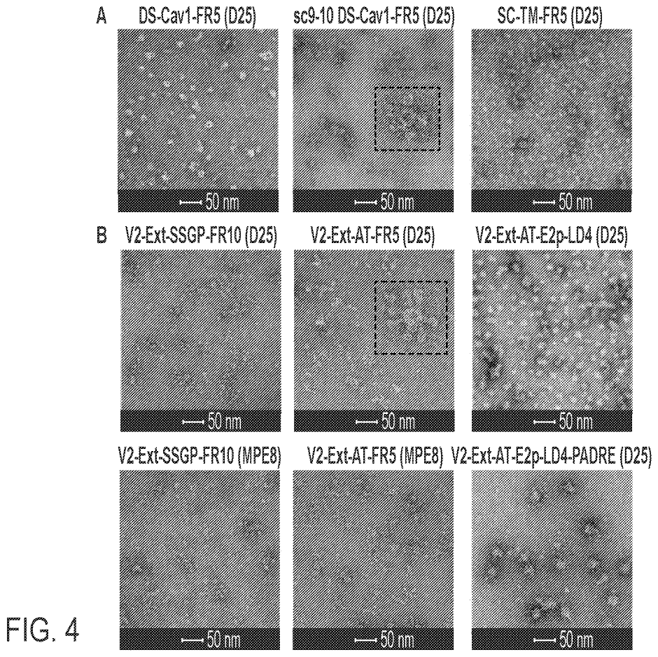

Negative-stain electron microscopy (nsEM) analysis was performed to characterize nanoparticles presenting RSV F trimers as described in Example 8 (FIG. 4). Two nanoparticle platforms, 24-meric ferritin and 60-meric E2p, were examined in this study.

The three representative RSV F designs were tested using the ferritin nanoparticle as a model display system (FIG. 4,A). A 5-GS linker was inserted between the C-terminus of F and the N-terminus of ferritin subunit. Among these three designs. sc9-10 DS-Cav1 was the performer with well-formed nanoparticles (FIG. 4, A, middle), whereas the ferritin-fusion constructs of DS-Cav1 and SC-TM failed to form nanoparticles or failed to form native-like F trimers (FIG. 4, A, left and right). Two redesigned RSV F trimers, V2-Ext-SSGP and V2-Ext-AT, were displayed on ferritin with 10-GS and 5-GS linkers, respectively (FIG. 4, B, columns 1 and 2). D25 and MPE8 antibody columns could be used to purify nanoparticles (FIG. 4, B. rows 1 and 2, columns 1 and 2). The EM analysis demonstrated that both redesigned F trimers could be displayed well on ferritin nanoparticles, with a shorter (5-GS) linker showing more visible and complete prefusion F spikes on the nanoparticle surface. Most importantly, our newly designed prefusion F spikes appeared to have visually recognizable difference in the shape compared to the sc9-10 DS-Cav1 trimer spikes, when they were both displayed on the ferritin nanoparticle. Specifically, the V2-Ext-AT F trimer spike adopted a “thumb”-like shape with solid surface, which is characteristic of a prefusion, closed trimeric spike, whereas the sc9-10 DS-Cav1 trimer spike appeared to be “lollipop”-like and hollow, which is indicative of an open conformation (FIG. 4, A, column 2, the box vs. FIG. 4, B, row 1, column 2, the box). Our EM data indicates that an inherently more stable F trimer without any features of RSV F metastability is crucial for the development of RSV F nanoparticle vaccines. It is also apparent that the all of the three existing, representative RSV prefusion F designs, DS-Cav1, optimized sc9-10 DS-Cav1, and SC-TM are not suitable for nanoparticle display. Lastly, we examined whether our newly designed RSV prefusion F trimers can be displayed on large, 60-meric nanoparticles with a locking domain (LD) and a build-in T-helper epitope. To this end, we expressed and purified two nanoparticle constructs, V1-Ext-AT-E2p-LD4 and V1-Ext-AT-E2p-LD4-PADRE, for EM analysis (FIG. 4, B, column 3). Consistently, well-formed nanoparticles with an array of “thumb”-like prefusion F trimers on the surface were observed for both constructs, confirming that the newly designed prefusion RSV F trimers can be displayed on the multilayered nanoparticle platforms as vaccine candidates.

The invention thus has been disclosed broadly and illustrated in reference to representative embodiments described above. It is understood that various modifications can be made to the present invention without departing from the spirit and scope thereof.

It is further noted that all publications, sequence accession numbers, patents and patent applications cited herein are hereby expressly incorporated by reference in their entirety and for all purposes as if each is individually so denoted. Definitions that are contained in text incorporated by reference are excluded to the extent that they contradict definitions in this disclosure.

1. An engineered soluble fusion (F) protein of a paramyxovirus, comprising an altered soluble F sequence that has modifications relative to wildtype soluble F sequence of the paramyxovirus, wherein the modifications comprise (1) substitutions of two or more negatively charged residues around the β23 strand (D486-A490) with polar or hydrophobic residues, (2) deletion of the P27 peptide (E110-R136), and (3) an engineered intra-protomer disulfide bond that is either within the F1 subunit or links the F2 and F1 subunits; wherein the amino acid numbering is based on human RSV A2 strain F protein (UniProt ID P03420).

2. The engineered soluble F protein of claim 1, wherein the paramyxovirus is RSV.

3. The engineered soluble F protein of claim 1, wherein the two or more negatively charged residues around the β23 strand are D486 and E487.

4. The engineered soluble F protein of claim 3, wherein the substitutions around the β23 strand comprises D486N/E487Q or D486L/E487L.

5. The engineered soluble F glycoprotein of claim 1, wherein the engineered disulfide bond is S155C/S290C, S62C/K196C, or E60C/K196C.

6. The engineered soluble F protein of claim 1, further comprising a linker moiety that replaces (1) the furin cleavage site(s) or (2) the unstructured C-terminus of F2 (Q98-R109) and part of the N-terminus of the fusion peptide (FP) (F137-V157).

7. The engineered soluble F protein of claim 6, wherein the replaced C-terminus of F2 comprises residues N104-R109 (104NNRARR109; SEQ ID NO:31).

8. The engineered soluble F protein of claim 6, wherein the replaced part of the N-terminus of the fusion peptide (FP) comprises F137-S146.

9. The engineered soluble F protein of claim 1, further comprising substitution of residue S215 or residue E92.

10. The engineered soluble F protein of claim 9, wherein residue S215 is replaced with P, and residue E92 is replaced with D, Q, another short and polar residue, or a hydrophobic residue.

11-12. (canceled)

13. The engineered soluble F protein of claim 1, further comprising V185P substitution, one or both of S46G and K462Q substitutions, an engineered intra-protomer disulfide bond S180C/S186C or A177C/T189C within the β3/β4 hairpin, or an engineered inter-protomer disulfide bond A149C/Y458C.

14-16. (canceled)

17. The engineered soluble F protein of claim 1, comprising the sequence shown in any one of SEQ ID NOs: 17-23, a conservatively modified variant thereof, or a substantially identical sequence thereof.

18. An engineered soluble fusion (F) protein of a paramyxovirus, comprising an altered soluble F sequence that has modifications relative to wildtype soluble F sequence of the paramyxovirus, wherein the modifications comprise an engineered disulfide bond that links a pair of β-sheet-forming amino acids in the β3/β4 hairpin or equivalent hairpin in the F1 subunit, wherein numbering of the hairpin is based on respiratory syncytial virus (RSV).

19. The engineered soluble F protein of claim 18, wherein the paramyxovirus is human RSV, wherein the engineered disulfide bond is between substituted residues S180C/S186C or A177C/T189C in the β3/β4 hairpin, and wherein the amino acid numbering is based on human RSV strain A2 having UniProt ID P03420.

20-34. (canceled)

35. An engineered soluble F protein of a respiratory syncytial virus (RSV), comprising a soluble RSV F sequence that is altered by (1) deletion of the P27 peptide (residues E110-R136), (2) a modification in the unstructured C-terminus of the F2 subunit (residues Q98-R109), and (3) a truncation of the N-terminus of the fusion peptide (residues F137-V157), wherein the amino acid numbering is based on human RSV strain A2 having UniProt ID P03420.

36-38. (canceled)

39. An paramyxovirus immunogenic composition, comprising the engineered soluble F protein of claim 1 that is displayed on the surface of a self-assembling nanoparticle.

40. The immunogenic composition of claim 39, wherein the self-assembling nanoparticle comprises a trimeric sequence, and wherein C-terminus of the immunogen polypeptide is fused to N-terminus of subunit sequence of the nanoparticle.

41. A polynucleotide sequence that encodes the immunogen polypeptide of claim 1.

42. A pharmaceutical composition, comprising the immunogen polypeptide of claim 1 or a polynucleotide encoding the immunogen polypeptide the polynucleotide of claim 41, and a pharmaceutically acceptable carrier.

43. A method of preventing or treating a paramyxovirus infection in a subject, comprising administering to the subject a therapeutically effective amount of the pharmaceutical composition of claim 42.

44. (canceled)
